SEQUENCE LISTING (2 sequences)

SEQ ID NO: 1 - RNA, 38 nt, Artificial Sequence
  oligonucleotide
acaagucauaccguacguucaggcauuuaaaaaaaaaa

SEQ ID NO: 2 - RNA, 36 nt, Artificial Sequence
  oligonucleotide
acaagucauaccguauuuuuuuuuuuuuuuaaaaaa

(19) United States
(12) Patent Application Publication
(10) Pub. No.: US 2019/0382790 A1
(43) Pub. Date: Dec. 19, 2019

(54) PERFLUOROCARBYLATED COMPOUNDS FOR THE NON-VIRAL TRANSFER OF NUCLEIC ACIDS

(71) Applicants: Konstanze Schäfer, BERLIN (DE); JEANETTE LIBERA-KORNER, BERLIN (DE)

(72) Inventor: Konstanze Schäfer, Berlin (DE)

(21) Appl. No.: 16/413,513

(22) Filed: May 15, 2019

Related U.S. Application Data
(63) Continuation of application No. 14/008,950, filed on Nov. 7, 2013, now abandoned.

(30) Foreign Application Priority Data
Mar. 31, 2011 (DE) 10 2011 016 334.4
May 9, 2011 (DE) 10 2011 101 361.3
Aug. 26, 2011 (DE) 10 2011 112 191.2
Oct. 20, 2011 (DE) 10 2011 117 390.4

(51) Int. Cl. C12N 15/85; A61K 47/54; A61K 49/00; A61K 47/52

(52) U.S. Cl. CPC C12N 15/85; A61K 47/52; A61K 49/0002; A61K 47/54

(57) ABSTRACT

An oligonucleotide or a nucleic acid covalently bonded to a perfluorocarbyl group has general formula ABE. A is a perfluorocarbyl group, including perfluorinated straight or branched aliphatic alkanes, perfluorinated straight or branched alkenes, perfluorinated straight or branched alkynes, and cyclic, optionally aromatic, perfluorocarbons, in which all of the H-atoms are substituted by F-atoms. B is a covalent bond, such as a covalent bond between the perfluorocarbyl group A and a C atom at the 2′ position of one or more sugars of the oligonucleotide or the nucleic acid, a covalent bond between the perfluorocarbyl group A and a C atom of a heterocyclic ring of one or more nucleobases of the oligonucleotide or the nucleic acid, or a covalent bond between the perfluorocarbyl group A and an O atom of one or more phosphate groups of the oligonucleotide or the nucleic acid. E is an oligonucleotide or a nucleic acid.

### RELATED APPLICATIONS

This application is a continuation application of U.S. application Ser. No. 14/008,950, filed Nov. 7, 2013, which is U.S. National Phase of International Application PCT/EP2012/055639, filed Mar. 29, 2012 and designating the U.S., which claims priority to German Application No. 10 2011 016 334.4, filed Mar. 31, 2011; German Application No. 10 2011 101 361.3, filed May 9, 2011; German Application No. 10 2011 112 191.2, filed Aug. 26, 2011; and German Application No. 10 2011 117 390.4, filed Oct. 20, 2011.

### FIELD OF THE INVENTION

A stable compound that overcomes drawbacks of viral gene transfer and is suited to non-viral gene transfer.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 30516449_1.TXT, the date of creation of the ASCII text file is May 15, 2019, and the size of the ASCII text file is 938 bytes.

### BACKGROUND OF THE INVENTION

Non-viral gene transfer is an important area of focus in basic research and in medicine. Possible applications arise particularly in relation to classic hereditary diseases and acquired genetic diseases (e.g., HIV, chronic infectious diseases, tumors, heart and circulatory diseases). In the past, attempts to establish gene therapy in medicine focused above all on viral vectors. However, they are associated with substantial drawbacks. The applications are not sufficiently safe and, what it more, trigger immune responses after one-time application in the body that make a second application impossible. Beyond that, incidents have been reported time and time again in which patients became very ill or even passed away as a result of the treatment.

One alternative to viral gene transfer could be non-viral gene transfer. However, all of the methods known thus far are so inefficient that they are not used in medicine. The non-viral transfer methods include all methods in which no viruses are involved.

The transfer of naked DNA or RNA has already been researched but offers few possible practical applications in the previous form, since transfusion is performed into open tissue or injection is performed into the bloodstream, and RNA and DNA are very fragile in relation to nucleases. What is more, the transfection rates are very low.

In order to work around the abovementioned problems, non-viral nucleic acid transfer by means of DNA or RNA complexes with cationic polymers (e.g., PEI, PEG, PLL, PLA) or with cationic lipids (e.g., CTAB, DOTMA, DOTAP) is gaining in importance. The positive charge of such molecules is used to neutralize the negative charge of the sugar-phosphate structure of the nucleic acid and facilitate absorption through the cell membrane into the cytoplasm of the cell. There are numerous patents on these methods. However, the investigational results in this context only mark the beginning of a trend. After all, besides the still unsatisfactory transfection rates, the toxicity of these polymers and lipids represents a crucial obstacle for the cell. Apart from that, these complexes tend to clump within the cytoplasm, since the biodegradability of the polymer is too low. The loading rates with DNA or RNA increase with the level of the positive charge of the polymer or the lipid. But it is precisely these highly positively charged molecules that have proven to be especially toxic to cells. In order to reduce the toxicity of these cationic polymers and lipids, they are increasingly being combined with hydrophilic polymers, although no outstanding improvement has been achieved in this way.

Apart from their low efficiency, previously known transport molecules for non-viral gene transfer have a second drawback in common: They remain in the cytoplasm after transport into the cell and accumulate there or react with cell molecules, or they have negative effects on the cell membrane.

Furthermore, research is being done in modifying nucleic acid building blocks so as to make them suitable for the non-viral transfer of nucleic acids. For instance, WO 2008/039254 and patent US 2010/0016409 describe RNA particles that are double-stranded in part or in whole or are present in other specific conformations and are optionally linked to other molecules. These RNA particles, which have greater stability than single-stranded mRNA due to their conformation, are proposed for non-viral gene transfer. The advantage of these molecules is that they are very small and can also pass through very fine capillary blood vessels. What is more, there is consequently hardly any danger of the clumping that often occurs with relatively large polymer complexes. One drawback of this molecule is that, besides the therapeutic sequences, additional sequences have to be built into the mRNA molecule that are intended to lead to the self-aggregation of certain areas, thus resulting in such RNA conformations as hair needle, nano-ring, quadratic and other structures occur. Although the RNA molecules have a longer life span within the organism than purely single-strand mRNA, the availability of these double-stranded conformations for translation to the ribosomes has not yet been demonstrated.

EP 1 800 697 B1 describes a modified mRNA whose G/C content is higher compared to the wild type and that at least one codon of the wild-type sequence that codes for a tRNA that is relatively rare in the cell is exchanged for a codon that codes for a tRNA that is relatively common in the cell. The mRNA modified in this way is additionally altered such that at least one nucleotide analog from the group consisting of phosphorthioate group, phosphoramidate group, peptide nucleotides, methyl phosphonate group, 7-deazaguanosine, 5-methyl cytosine and inosine is incorporated which have already been used in several other RNA methods (siRNA). The method is described for sequence-altered mRNAs from original wild-type peptides.

Document WO 99/14346 also describes an mRNA stabilized through sequence modifications, particularly with a lower C- and/or U-content through base elimination or base substitution.

The patents U.S. Pat. Nos. 5,580,859 and 6,214,804 describe transient gene therapy constructs that are composed of an DNA expression vector.

WO 02/098443 describes mRNAs that code for a biologically active peptide that is either not formed or is not formed accurately in the patient to be treated, and hence does not trigger an immune response.

Another development in the area of non-viral gene therapy is “microbubble” technology, in which stabilized protein microspheres filled with nucleic acid (Kausik Sarkara et al., J. Acoust. Soc. Am. 118, Jul. 1, 2005, pages: 539-550) or sugar microspheres (Schlief et al., Ultrasound in medicine & Biology, Volume 22, Issue 4, 1996, pages 453-462) are additionally filled with ultrasound gases. It had been observed that ultrasound contrast media lead to an intensification of cavitation as a result of which the cell membrane is transiently permeabilized (Tachibana et al., Echocardiography. 2001 May; 18(4):323-8. Review). This lead to an increased absorption of the non-viral gene transfer constructs into the cell. Nonetheless, viral gene transfer was not efficiently achieved.

The ultrasound method with contrast media is also being increasingly used in order to increase the efficiency of viral gene transfer (Blomley, September 2003, Radiology, 229, 297-298).

In addition to series of other gases, perfluorocarbon gases have proven to be especially suitable for “microbubble” technology. As a result of their highly lyophilic properties and their extremely low surface tension, they are highly suited to disturb the integrity of the cell membrane and thus allowing substances to pass through. These gases are pure perfluorocarbons that are not bound in some way with other components. Experiments on non-viral gene transfer by using pure perfluorocarbons have shown, however, that the nucleic acids diffuse away from pure perfluorocarbons before entering the cell. In this way, nucleic acids can only be incorporated as a result of random events.

All of the previously described solutions are still distant the efficiency of viral gene transfer. There is thus great interest in the development of a non-viral transfer system for nucleic acids into the cell which firstly, have an effectiveness which is at least equal to the effectiveness of viral gene transfer, and secondly for which the components do not accumulate in the cell, do not react with the cell molecules, and do not have a harmful effect on the cell membrane.

### SUMMARY OF THE INVENTION

Accordingly, an object of the present invention is to provide a stable compound that overcomes the drawbacks of viral gene transfer and is suited to non-viral gene transfer.

This object was achieved accordingly through the provision of a compound for the non-viral transfer of nucleotide building blocks with the features of claim 1.

The compound according to the invention comprises a structure of general formula (I):

A-B-C(F′, G′)-D-E-F-G-A′ (I)

or a structure of general formula (II):

A-B-C(F′, G′)-D-B-E-F-G-A′ (II)

wherein:

A is at least one substituent selected from the group of the perfluorocarbyl (PFC), perfluorosilyl and/or other perfluorocarbylated substituents,

B is at least one predetermined breaking point in the form of a physically, chemically or enzymatically severable bond,

C is absent or at least one linker,

D is absent or at least one spacer,

E is at least one structure selected from nucleobases, nucleosides, nucleotides, oligonucleotides, nucleic acids, modified nucleobases, modified nucleosides, modified nucleotides, modified oligonucleotides, modified nucleic acids, peptide nucleic acid monomers, peptide nucleic acid oligomers and peptide nucleic acids or other nucleic acid analogs,

F, F′ is absent or at least one ligand or a recognition sequence,

G, G′ is absent or at least one marker,

A′ is absent or has the meaning of A,

and wherein the following compounds or compounds comprising the following cations:

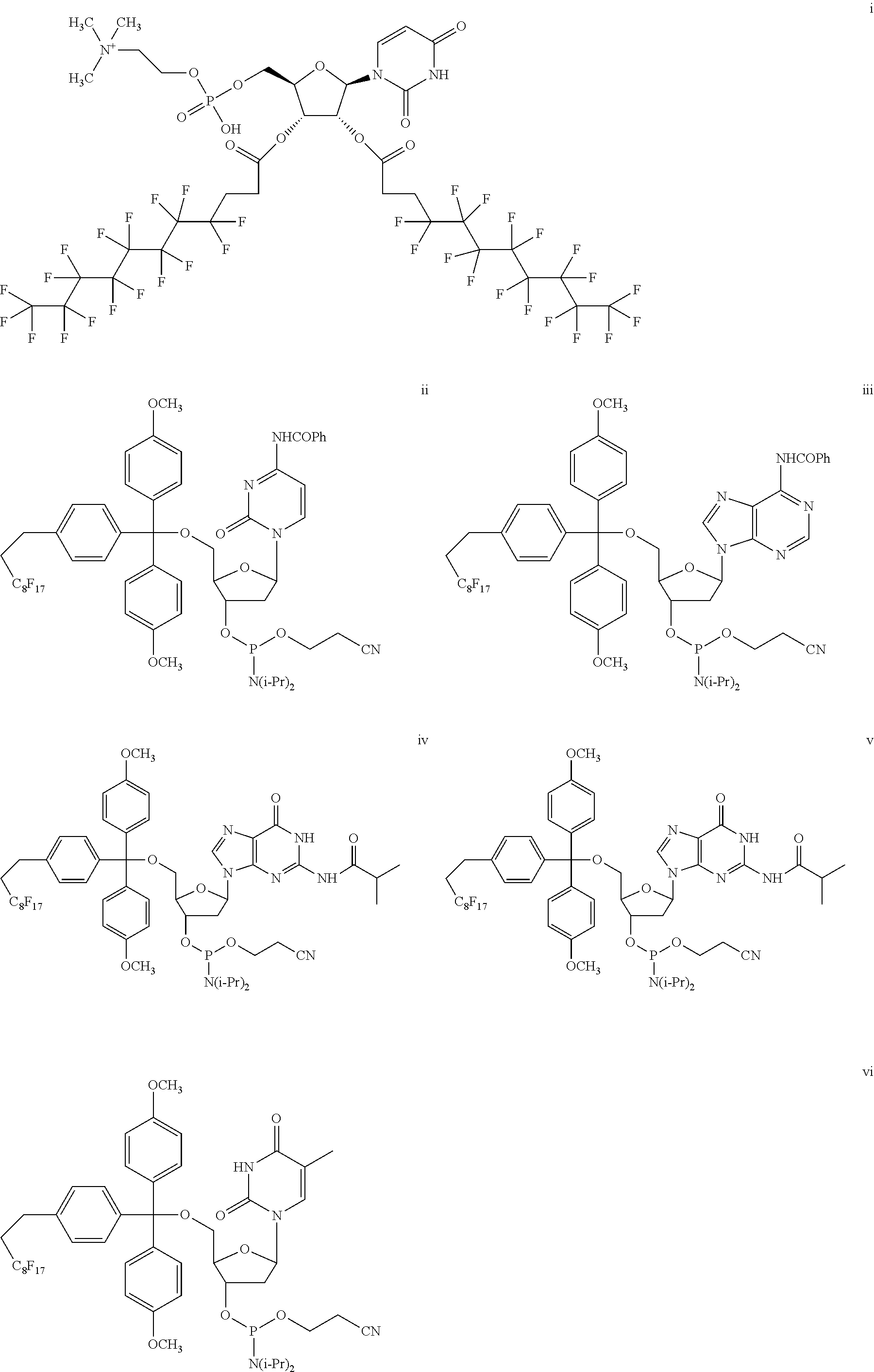

are excluded.

The structures A, B, C, D, E, F, F′, G, G′ and A′ are preferably each linked together via covalent bonds. However, it is conceivable for the individual structures of the compound according to the invention to be linked together in whole or in part by ionic bonds.

### DETAILED DESCRIPTION OF THE PREFERRED EMBODIMENT

Accordingly, a new, promising type of compound for non-viral gene transfer are compounds particularly including perfluorocarbyl groups (PFCs) and, for example, nucleic acid structures that are linked together via a predetermined breaking point, so-called perfluorinated nucleic acid containing compounds.

The advantages of perfluorinated nucleic acid containing compounds are as follows:

1) As a result of their highly lyophilic properties under physiological conditions (more lyophilic than fatty acids) and even more due to their extremely low surface tension, these molecules attach quickly to the surface of the cell membrane. There, they are absorbed into the cell through regularly occurring processes such as pinocytosis, phagocytosis, endocytosis or endocytosis-independent paths. Here, the added PFCs groups are the transport system into the cell for the otherwise not readily absorbable nucleic acids.

2) Due to their strong C—F bonds, PFC groups are very inert and do not react with cell molecules.

3) After breaking off from the nucleic acid structure, PFC groups are small, uncharged lyophilic molecules which, depending on the concentration gradient, can exit the cell passively.

4) Through the medical application of perfluorocarbons as oxygen carriers/blood substitutes in humans, it has been shown that perfluorocarbons are excreted from the body through the lungs, kidneys and skin.

5) Given that they have already been approved as blood substitutes and as contrast media, the medical approval of perfluorocarbons for non-viral gene transfer may be easier.

6) Nucleic acids with perfluorocarbyl groups exhibit significantly greater absorption into the cell than other substances of non-viral gene transfer and are a true alternative to viral gene transfer.

7) Unlike in viral gene transfer, nucleic acids with perfluorocarbyl groups do not generate any immune response of the body and can be used as often as desired.

8) In connection with an mRNA transfer, dosing can be achieved due to the limited life span of the mRNA and the unlimited repeatability of the transfer.

Pure perfluorocarbons are originally known from the high-performance lubricants industry. In the pharmaceuticals sector, they have previously been used as blood substitutes above all due to their high degree of oxygen solubility, or even as contrast media. It has also been shown that, in ultrasound applications with non-viral gene transfer microbubbles that were filled with gaseous pure perfluorocarbons, the efficiency of the gene transfer was increased compared to other gases.

However, pure perfluorocarbons are hardly suitable for the non-viral gene transfer of nucleic acids into the cell, since nucleic acids do not adhere to them and can therefore only be taken along by random events. A true bond is needed between the perfluorocarbon group and nucleic acids which can be split at a predetermined breaking point.

Accordingly, the compound according to the invention is characterized by the absorption of the perfluorocarbyl substituted nucleic acids into the cell, the breaking at the predetermined breaking point between nucleic acid and perfluorocarbyl group, the release of cleavage products (nucleic acids on the one hand and molecules derived from the perfluorocarbyl groups on the other) into the cytoplasm, and the subsequent diffusion or the active discharging of the molecules derived from the perfluorocarbyl groups from the cell. An endocytosis-independent absorption of the perfluorocarbyl substituted nucleic acids is also possible.

As explained above, perfluorocarbyl substituted nucleic acids are both charged and very lyophilic molecules. The secondary and tertiary structure of these molecules is very well suited to destabilize the cell membrane and being absorbed into the cell. Under physiological conditions, perfluorocarbons are even more lyophilic than fatty acids and have an extremely low surface tension, which enables the molecule to extend over a large surface of the cell membrane.

By means of a predetermined breaking point between the nucleic acid structure and the perfluorocarbylated portion of the compound, after entering the cell, the perfluorocarbylated portion of the nucleic acid is split off. This usually occurs via acid-labile predetermined breaking points. The increased reduction potential in the cytoplasm and, more so, the low pH value in the endosomes (down to pH=4.5) create the conditions for the hydrolysis thereof.

The predetermined breaking points for this system are sought out such that the cleavage products experience no or little molecular alteration. “Traceless” predetermined breaking points that leave behind an unchanged nucleic acid and a perfluorocarbyl containing molecule, that has obtained its extremely lypophilic and non-polar nature, are very suitable for this. One example of such a predetermined breaking point is shown in the following diagram 1:

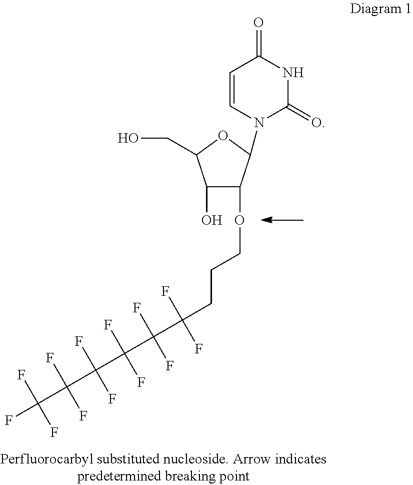 [SMILES: O=c1ccn(C2OC(CO)C(O)C2OCCCC(F)(F)C(F)(F)C(F)(F)C(F)(F)C(F)(F)C(F)(F)F)c(=O)[nH]1]

The nucleic acids released into the cytoplasm are freely accessible for the cell. They can have their site of action in the cytoplasm such as, for example, mRNA, siRNA, microRNA, aptamers, antisense RNA and others, or they can be transported into the nucleus, such as DNA with or without nucleus localization sequence, antisense oligonucleotides or individual nucleotides and nucleosides.

The perfluorocarbyl containing molecules formed by cleavage at the corresponding predetermined breaking point also released into the cytoplasm are uncharged, lyophilic and relatively small. These characteristics are the conditions for free diffusion along the concentration gradient through the cell membrane. Exocytosis or another release path out of the cell is also possible. Perfluorocarbyl containing molecules are extremely inert and do not react with cell molecules. As long as their molecular structure remains relatively unchanged, they also do not attach to lipids. It is known from the medical use of perfluorocarbons as blood substitutes that they are excreted from the body via the lung and kidney function as well as through the skin.

The perfluorocarbyl substituted nucleic acids can also be linked with fluorescent dyes in order to follow their path in the cell. By linking with specific ligands or other recognition sequences, the system can be set up for the treatment of special cell types. In principle, the transfer system comprising perfluorocarbyl substituted nucleic acids can be used for any application in which nucleic acids or modified nucleic acid analogs are to be transported into a cell.

In one embodiment of the present compound, the at least one structure A is selected from the group of the perfluorocarbyl groups (PFCs) containing straight or branched acyclic or cyclic, polycyclic or heterocyclic aliphatic alkyls, alkenyls, alkynyls, aromatic substituents or combinations of these substituents in which all of the H-atoms are substituted by F-atoms which can optionally also contain at least one non-fluorinated or partially fluorinated substituent in the form of one or more functional groups, aliphatic chains or heteroatoms, containing particularly Br, I, Cl, H, Si, N, O, S, P or these in conjunction with one or more additional functional groups.

It is also preferred that A be selected from the group of substituents derived from perfluorocarbyl (PFCs) containing C₁-C₂₀₀, preferably C₁-C₁₀₀, particularly preferably C₁-C₅₀, very preferably C₁-C₃₀, most preferably C₁-C₂₀ alkyl, alkenyl or alkynyl which can be linear, branched, cyclic, polycyclic or heterocyclic, C₆-C₅₀, preferably C₆-C₃₀, particularly preferably C₁-C₂₀ aryl or heteroaryl groups.

Typically, in relation to the present invention, A structures can be selected from the PFC group containing —(CnₙF₍₂ₙ₊₂₎₋₁) where n≥1, preferably n=1-20, for example —CF₃, —C₂F₅, —C₃F₇, —C₄F₉, —C₄F₁₁, etc., —(Cnₙ F₂ₙ₋₁) where n≥2, preferably n=2-20, for example —C₂F₃, —C₃F₅, —C₄F₇, etc., —(CnₙF₍₂ₙ₋₂₎₋₁) where n≥2, preferably n=2-20, for example —C₂F, —C₃F₃, —C₄F₅, —C₅F₇, etc.

To manufacture the compound according to the invention, it is sensible to use perfluorinated compounds that have suitable functionality in order to enter into a covalent bond with the other structures such as B and E. This functionality of the perfluorinated compound used as the starting substance enables, in particular, addition, substitution, esterification, etherification, condensation, etc. Such ligation reactions are known to the person skilled in the art. Preferred functionalities for the perfluorocarbyl containing substituent are selected from the list containing halogen alkanes, hydroxyl, ether, amino, sulhydryl, aldehyde, keto, carboxyl, ester and acid amide groups, groups with radicals or ions, and from the following list of compounds carboxilic acids, peroxycarboxylic acids, thiocarboxylic acids, sulfonic acids, sulfinic acids, sulfenic acids, sulfoxides, carboxylic acid salts, sulfonic acid salts, sulfinic acid salts, sulfenic acid salts, carboxylic acid anhydrides, carboxylic acid esters, sulfonic acid esters, acyl halides, sulfonyl halides, carboxylic acid amides, sulfonamides, carboxylic acid hydrazides, nitriles, aldehydes, thioaldehydes, ketones, thioketones, oximes, alcohols, phenols, thiols, amines, imines, hydrazines, ethers, esters, thioethers, thioesters, hydrogen halides, nitro compounds, nitroso compounds, azo compounds, diazo compounds, diazonium salts, isocyanates, cyanates, acid amides and thioethers or compounds that can be reactive due to their multiple bond. Especially preferred are building blocks substituted by heteroatoms such as Br, I, Cl, H, Si, N, O, S, P, hydroxyl, amino, carboxyl groups, haloalkyl, carboxylic acid amines, alcohols, hydrazines, isocyanates, thiocyanates and acid amides.

Preferably, as a starting material for the preparation of structure A, according to the invention, a substance is used that is suitable for nucleophilic substitution. The starting material for the preparation of structure A has a nucleophilic leaving group that is readily recognizable for a person skilled in the art. Accordingly, additional preferred starting material for preparation of A-structures can be based on any one of the following starting substances:

F(CF₂)ₙX, where n=1-50, preferably n=1-10, and X=Br, I, Cl, H or X=Si, N, O, S, P in conjunction with a functional group, particularly C₈F₁₇I, C₈F₁₇Br;

F(CF₂)ₙ(CH₂)ₘX, where n=1-50, preferably n=1-10, m=1-26, preferably m=1-6 and X=Si, N, O, S, P, Br, I, H;

F(CF₂)ₙ—O_{b}—CH═CH₂, where n=1-50, preferably n=1-10, and b=0 or 1, preferably b=0;

C₆F₁₃CH₂CH₂MgI, (C₆F₁₃CH₂CH₂)₃SnPh, (C₆F₁₃CH₂CH₂)₃SnBr, (C₆F₁₃CH₂CH₂)₃SnH;

C₂F₅I, C₃F₇Br, C₄F₉I, C₅F₁₁Br, C₆F₁₃Br, C₈F₁₅Br, C₁₀F₁₇I,

C₄F₉CH═CHC₄F₉, C₈F₁₆C₁₂, C₁₀F₁₉N, C₆F₁₉Br, C₉F₂₁N, C₁₀F₂₁Br, C₁₁F₂₂N₂O₂, C₆F₁₃CH═CHC₆F₁₃, C₁₂F₂₇N, C₁₂F₂₇N, C₁₆F₂₅Br;

C₈F₁₇I, C₈F₁₇Br.

Additional preferred starting substances for preparation of A, according to the present invention, are selected from the group of perfluorohydrocarbyl containing building blocks:

perfluorocarbyl cholesteryl and adamantyl building blocks, perfluorocarbyl cis-eicosenoic, perfluorocarbyl aromatic building blocks, perfluorocarbyl pyrenes, perfluorocarbyl glycerides; and

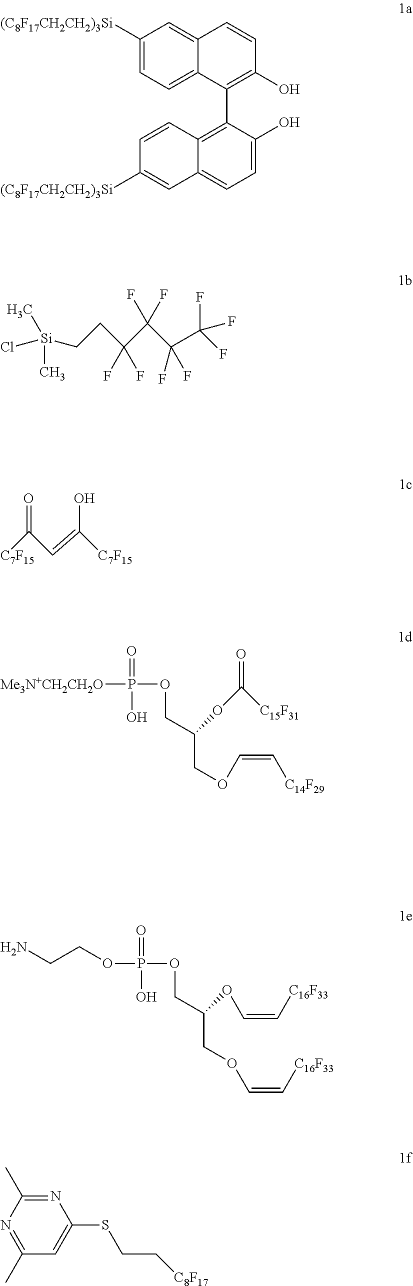

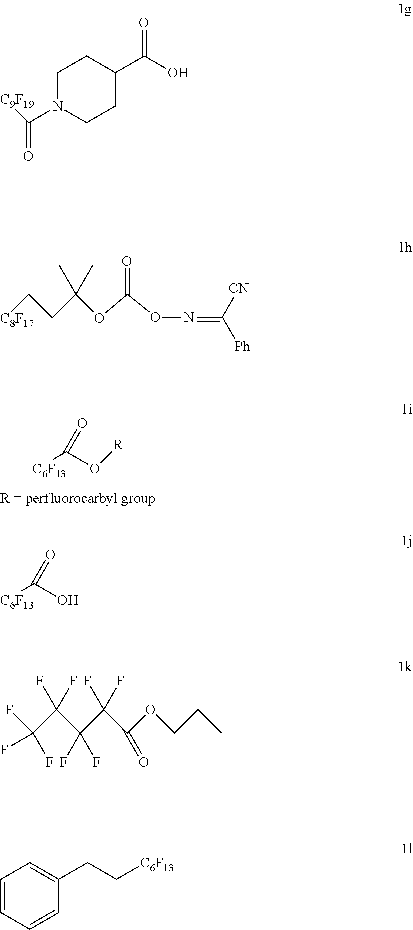

for linking in the form of an ionic bond, the following perfluorocarbyl derivatives can be used, for example:

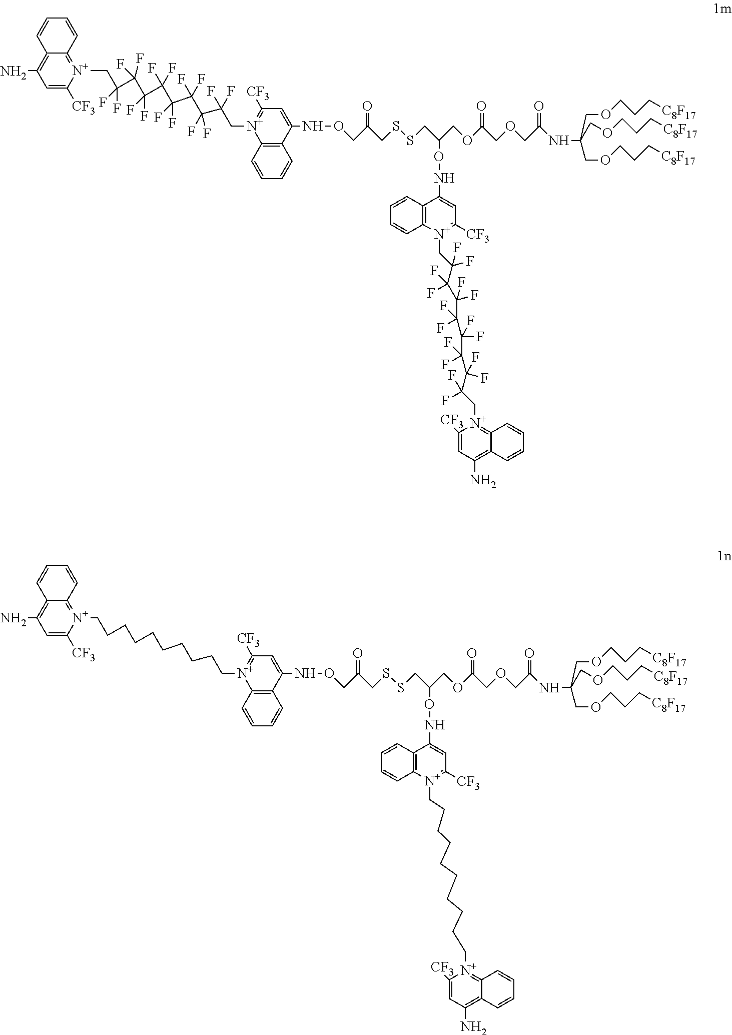

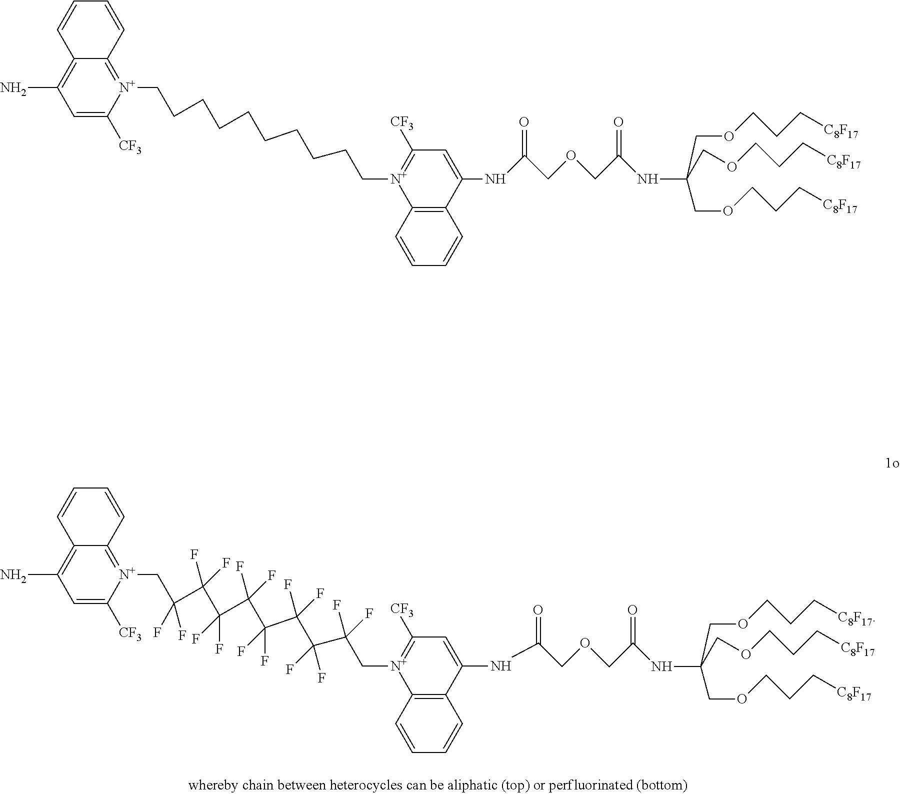

Another embodiment of the present invention is that at least one starting material for the preparation of the structure A is selected from the perfluorosilyl substituents containing straight or branched acyclic or cyclic, polycyclic or heterocyclic aliphatic silanes in which all H-atoms are substituted by F-atoms, which optionally and additionally contain non-fluorinated or partially fluorinated substituents with one or more functional groups or heteroatoms, particularly Br, I, Cl, H, Al, N, O, S, P, or these in combination with one or more additional functional groups.

Preferably, perfluorosilyl substituents from the list Si1-Si200, preferable Si1-Si100, particularly preferably Si1-Si50, very preferably Si1-Si30, most preferably Si1-Si20 perfluorocarbylated silyl containing building blocks are used. The perfluorosilyl substituents are also functionalized with suitable substituents as in the case of the perfluorocarbyl groups.

In yet another embodiment, the at least one starting material for the preparation of the structure A is selected from the group of other perfluorocarbylated substituents being based on substituents that are selected from among NF₃, N₂F₄, SNF₃, CF₃SN, SF₄, SF₆, or perfluorocarbylated nitrogen-sulfur substituents.

It is also preferred that the present compound contains two or more A structures selected from the group of perfluorocarbyl (PFC), perfluorosilyl and other perfluorocarbylated substituents.

Typically, the above described functionalized perfluorocarbylated substituents can be integrated several times into fragment A. Reference is made to the following molecules as examples:

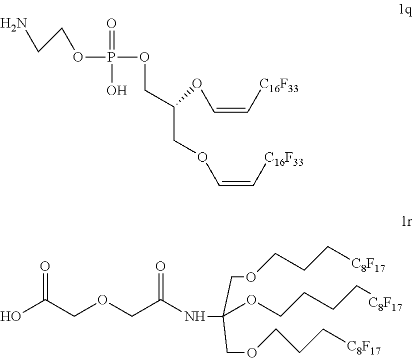

The linking of these molecules to the other structures of the compound according to the invention can be carried out via NH₂ groups or OH groups or other suitable groups.

As a variant of the compound, the at least one predetermined breaking point B may be an acid-labile group, particularly in the form of a glycosidic bond, at least one disulfide bridge, at least one ester group, ether group, peptide bond, imine bond, hydrazone bond, acylhydrazone bond, ketal bond, acetal bond, cis-aconitrile bond, trityl bond, beta-D-glucosylceramide, and/or dithiothreitol.

Predetermined breaking points between perfluorocarbyl containing groups and nucleic acid structures have two important functions. Firstly, the predetermined breaking points serve the purpose of so-called “leakage.” Here, the perfluorocarbyl containing nucleic acid compounds attach to the endosome membrane of cells and are then be released from these endosomes. This occurs by destruction of the integrity of the membrane. Secondly, the perfluorocarbyl containing compounds have to be split off in order to make the nucleic acid derivatives available to the cell. Here, the acid-labile predetermined breaking points such as the gylycosidic bonds, disulfide bridges, esters or ethers listed above are hydrolyzed (break) under acidic conditions chemically or by hydrolases/esterases, for example at the 2′ position of the nucleotide or at other locations.

More complex predetermined breaking points can be present in the form of specific substrates for enzymes, in the form of pH- and photosensitive lipid functions, in the form of molecules that are released by ultrasound action or temperature-controlled lipid modifications, as well as in the form of other bonds that can be split under physiological conditions.

Reference is made to the following predetermined breaking points shown in the following examples:

Plasmalogen perfluoride, cleavable by light:

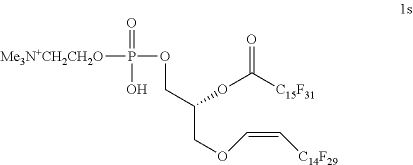

pH-sensitive perfluorocarbylated vinyl ether functions:

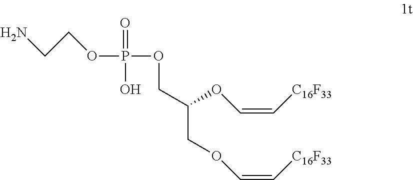

Orthoesters cleavable by rearrangement:

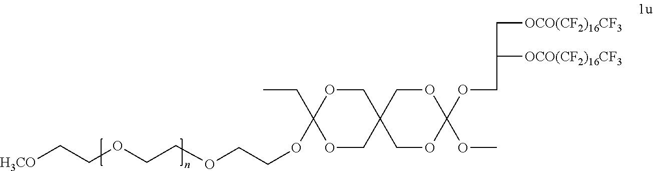

In one embodiment of the present invention, the linker group C, is selected from derivatives of straight or branched acyclic or cyclic, polycyclic or heterocyclic aliphatic alkanes, alkenes, alkynes, aromatic groups or combinations of these groups with functional groups.

The linker C used is preferably used in order to generate one or more bond sites for other, additional groups such as, for example, markers G′ and/or ligands F′ or other recognition sequences.

Preferably, the linker C is selected from the following substituents: haloalkyl, hydroxyl, alkoxyalkyl, amino, sulfhydryl, aldehyde, keto, carboxyl, ester and acid amide groups, groups with radicals or ions, and substituents derived from the following: carboxylic acids, peroxycarboxylic acids, thiocarboxylic acids, sulfonic acids, sulfinic acids, sulfenic acids, sulfoxides, carboxylic acid salts, sulfonic acid salts, sulfinic acid salts, sulfenic acid salts, carboxylic acid anhydrides, carboxylic acid esters, sulfonic acid esters, carboxylic acid halides, sulfonic acid halides, carboxylic acid amides, sulfonic acid amides, carboxylic acid hydrazides, nitriles, aldehydes, thioaldehydes, ketones, thioketones, oximes, alcohols, phenols, thiols, amines, imines, hydrazines, ethers, esters, thioethers, thioesters, hydrogen halides, nitro substituents, nitroso substituents, azo substituents, diazo substituents, diazonium salts, isocyanates, cyanates, isocyanates, thiocyanates, isothiocyanates, hydroperoxides, peroxides, or substituents that can be reactive due to their multiple bond, or functionalized perfluorohydrocarbyl substituents containing iodine, bromine or sulfur atoms, carbamates, thioether or disulfide groups, glycerol, succinyl glycerol, phosphateas well as functionalized perfluorohydrocarbyl substituents containing other groups or atoms via which a link can be established to nucleosides, nucleotides, oligonucleotides, nucleic acids, modified nucleosides, modified nucleotides, modified oligonucleotides, modified nucleic acids, peptide nucleosides, peptide nucleotides, peptide oligonucleotides, peptide nucleic acids or pharmaceutical substances.

Preferred linkers C are based on hydroxyl or amino groups, carboxyl groups, esters, ethers, thioethers, thioesters, carboxylic acid amides, substituents with multiple bonds, carbamates, disulfide bridges and hydrazides, haloalkyl, sulhydryl, aldehyde, keto, carboxyl, ester and acid amide groups, thiols, amines, imines, hydrazines, or disulfide groups, glycerol, succinyl glycerol, orthoesters, phosphoric acid diesters and vinyl ethers, ester and ether groups and disulfide bridges being very especially preferred.

As described above, the linker C can be used to link other markers G′ and/or ligands F′ or other recognition sequences which substantially comprise the same groups as the ligand F and marker G defined below but nonetheless differ from these by the arrangement within the present compound.

Suitable examples of the marker G′ are fluorescent dyes such as Dil, DilC, DiO, fluoresceins, rhodamines, oxacines, fuchsines, pyronines, acridines, auramines, pararosanilines, GFP, RFP, DAPI or peroxidase dyes such as ABTS.

Ligands and recognition sequences are required for specific transfer in certain cell types, since they bind to receptors on the cell surface and thus enable specific entry into the cell. Ligands are generally bound to the compound via accessible side or terminal amino groups. Bonds via other groups are possible, however.

Transferrin, folic acid, galactose, mannose, epidermal growth factor, RGD peptides, biotin, and other substances can be used here as suitable ligands F′. Examples of recognition sequences are nucleus localization sequences or sequences for endocytosis-independent absorption.

In another variant of the present invention, the spacer D is selected from straight or branched aliphates with one or more functional groups, allowing to use the spacer D as linker C.

According to the present invention, a spacer is used to prevent steric impediments between the molecules within the compound or is used in order to weaken the negative charge of the fluorine containing groups to other molecule areas. Especially preferred, the spacer D is derived from the group of the fatty acid alcohols, fatty acid diols and fatty acid polyols.

In a preferred embodiment of the present invention, nucleobases are used as structure E, which are selected from adenine, guanine, hypoxanthine, xanthine, cytosine, uracil, thymine, modified nucleobases such as 5-bromouracil, 5-fluorouracil, zidovudines, azidothymidines, stavudine, zalcitabine, diadenosine, idoxuridine, fluridine and ribavirin, azidothymidine, zidovudine, 5-methyluracil, 5-methylcytosine, 5-fluorocytosine, 5-bromocytosine, 2-aminopurine and “spiegelmers” thereof, the nucleobases adenine, guanine, cytosine, uracil and thymine being especially preferred.

If nucleosides are used to make structure E, as is preferred, then the nucleosides are selected from adenosine, guanosine, cytidine, 5-methyluridine, uridine, deoxyadenosine, deoxyguanosine, thymidine, deoxyuridine, deoxycytidine or modified nucleosides such as 2-thiocytidine, N4-acetylcytidine, 2′-O-methylcytidine, 3-methylcytidine, 5-methylcytidine, 2-thiouridine, 4-thiouridine, pseudouridine, dihydrouridine, 5-(carboxyhydroxymethyl)-uridine, 5-carboxymethylaminomethyl-uridine, 5-methylaminomethyluridine, 5-methoxy-carbonylmethyl-uridine, 5-methoxyuridine, 2′-O-methyluridine, ribothymidine, 1-methyladenosine, 2-methyladenosine, N6-methyladenosine, N6-isopentenyladenosine, 2′-O-methyladenosine, inosine, 1-methylinosine, 1-methylguanosine, N2-2-methylguanosine, N2-2,2-dimethylguanosine, 7+-methylguanosine, 2′-O-methylguanosine, queuosine, β-D-galactosylqueuosine, β-D-mannosyl-queuosine, archaeosine, 2′-O-ribosyladenosinphosphate, N6-threonylcarbamoyladenosine, lysidine, nicotinic acid, riboflavin and pantothenic acid, NADPH, NADH, FAD, coenzyme A, and succinyl coenzyme A, puromycin, aciclovir, ganciclovir and “spiegelmers” thereof, the nucleosides adenosine, guanosine, uridine, cytidine, deoxyadenosine, deoxyguanosine, deoxycytidine, deoxythymidine being especially preferred.

In another preferred embodiment of the present compound, nucleotides are used to make structure E that are selected from the group containing AMP, GMP, m5UMP, UMP, CMP, dAMP, dGMP, dTMP, dUMP, dCMP, cAMP, cGMP, c-di-GMP, cADPR, ADP, GDP, m5UDP, UDP, CDP, dADP, dGDP, dTDP, dUDP, dCTP, ATP, GTP, m5UTP, UTP, CTP, dATP, dGTP, dTTP, dUTP, dCTP or modified nucleotides that originate from the above-described building blocks, nucleotides with modifications on the sugar-phosphate structure, zwitterionic oligonucleotides as well as nucleotides in which the phosphate has been replaced by methyl phosphonate or a dimethyl sulfone group, AMP, GMP, m5UMP, UMP, CMP, dAMP, dGMP, dTMP, dUMP, dCMP being especially preferred.

It is further preferred to use deoxyribonucleic acids, ribonucleic acids and modified nucleic acids to make structure E, such as, for example, nucleoside phosphorothioates, zwitterionic nucleic acids, nucleic acids in which the phosphate has been exchanged for a methyl phosphonate or dimethyl sulfone group, bridged nucleic acids (locked nucleic acids), “spiegelmers,” nucleic acids in which the ribose-phosphodiester backbone has been exchanged for various polymeric constructs, such as a hexitol-based backbone strand or a nucleic acid analog based on glycerin units), morpholino oligonucleotides, phosphorthioate deoxyribonucleic acid, cyclohexene nucleic acids, N3′-P5′-phosphoramidates, tricyclo-deoxyribonucleic acids, morpholino phosphoramidate nucleic acids, threose nucleic acids, with nucleoside phosphorothioates, phosphorthioate deoxyribonucleic acid being especially preferred.

It is also preferred to make structure E by using monomers of the peptide nucleic acids such as (Fmoc)-adenine-(Bhoc)-OH, (Fmoc)-cytosine-(Bhoc)-OH, (Fmoc)-guanine-(Bhoc)-OH, (Fmoc)-thymine-(Bhoc)-OH or peptide nucleic acids in which the complete ribose phosphodiester backbone has been replaced by a peptidic, achiral backbone that is based on N-(2-amino-ethyl)glycine subunits in which the bases are linked to the backbone via a carboxymethylene unit, with (Fmoc)-adenine-(Bhoc)-OH, (Fmoc)-cytosine-(Bhoc)-OH, (Fmoc)-guanine-(Bhoc)-OH, and (Fmoc)-thymine-(Bhoc)-OH being especially preferred.

If single-strand or double-strand oligonucleotides and nucleic acids are used to make structure E, as is preferred, then they can have a length of 2 base pairs of up to greater than 1,000,000 bp, the following length ranges each being preferred: 10 to 50 bp, 15 to 25 bp, 25 to 200 bp, 25 to 100 bp, 200 to 300 bp, 200 to 500 bp, 500 to 1500 bp, 800 to 1300 bp, 1500 to 20,000 bp, 1500 to 5000 bp, 3000 to 8000 bp, 20,000 to 1,000,000 bp or 20,000 to 50,000, oligonucleotides with a length between 10 to 50 bp, 200 to 500 bp and 500 to 1500 bp being very especially preferred.

In a variant of the compounds of the invention, E is provided with functional groups selected from groups containing hydrazides, haloalkyl, hydroxyl, ether, amino, sulhydry-, aldehyde, keto, carboxyl, ester and acid amide groups, groups with radicals or ions, and substituents selected and derived from carboxylic acids, peroxycarboxylic acids, thiocarboxylic acids, sulfonic acids, sulfinic acids, sulfenic acids, sulfoxides, carboxylic acid salts, sulfonic acid salts, sulfinic acid salts, sulfenic acid salts, carboxylic acid anhydrides, carboxylic acid esters, sulfonic acid esters, carboxylic acid halides, sulfonic acid halides, carboxylic acid amides, sulfonic acid amides, carboxylic acid hydrazides, nitriles, aldehydes, thioaldehydes, ketones, thioketones, oximes, alcohols, phenols, thiols, amines, imines, hydrazines, ethers, esters, thioethers, thioesters, hydrogen halides, nitro substituents, nitroso substituents, azo substituents, diazo substituents, diazonium salts, isocyanates, cyanates, isocyanides, thiocyanates, isothiocyanates, hydroperoxides, peroxides, or groups that can be reactive due to their multiple bond, or functionalized perfluorohydrocarbyl groups with iodine, bromine or sulfur atoms, carbamates, thioether or disulfide groups, glycerol, succinyl glycerol, phosphate groups or other functional groups that allow a bonding to a functionalized perfluorohydrocarbyl containing fragment.

It is understood that the functional groups serves as as additional substituents on the nucleotide fragment. For example, an NH₂ group can be introduced at the 2′H position of the deoxyribose of a nucleoside or nucleotide in order to prepare this site for perfluorcarbyl derivatisation, which was originally not usable for perfluorcarbyl derivatisation. An SH group can also be introduced, for example, at the 2′OH position of the ribose in order to create a disulfide bridge later.

Preferred substituents or functional groups on structure E are —OH, —NH₂ and —SH groups, hydrazides, halogen alkanes, sulhydryl, aldehyde, keto, carboxyl, ester and acid amide groups, ethers, thioesters, and thioethers.

According to the present invention, the ligand F is preferably derived from transferrin, folic acid, galactose, lactose, mannose, epidermal growth factor, RGD peptides, biotin, and other substances that enable a specific entry of the compounds of the invention into the cell.

According to the present invention the marker G is preferably derived from fluorescent dyes such as Dil, DilC, DiO, fluoresceines, rhodamines, oxacines, fuchsines, pyronines, acridines, auramines, pararosanilines, GFP, RFP, DAPI, peroxidase dyes such as ABTS and other substances that enable the compound of the invention to be tracked during metabolism.

The compound of the invention is particularly suitable and can be used for the non-viral transfer of at least one parent molecule derived from E into at least one cell of a eukaryotic organism, particularly of animals or humans.

Especially preferably, the compound of the present invention is used in the form of a pharmaceutical composition with at least one surface-active substance, suitable surface-active substances being, for example, poloxamers, lecithins or other cell-tolerated surfactants.

The pharmaceutical composition can be present in the form of an emulsion, dispersion, suspension or solution, particularly with an average particle size between 2 nm and 200 μm, preferably between 20 nm and 400 nm, particularly preferably at 50 nm. The particle size can vary depending on the application. Through suitable methods, for example a sonicator, atomization or solvent method, the desired particle size can be set that is favorable for absorption into the cell. The present pharmaceutical composition is also suitable and can be used for the non-viral transfer of at least one parent molecule derived from group E into at least one cell of a eukaryotic organism, particularly of animals or humans.

The present compounds can be manufactured and modified in various ways. In particular, the linking of the perfluorocarbylated substituents such as perfluorohydrocarbyl (PFCs) and/or perfluorosilyl substituents to E can be achieved at different, accessible positions of the structure E.

In terms of the present application, a perfluorination site or position is to be understood particularly as the place in structure E in which the linking of E to the perfluorocarbylated group A or A′ preferably occurs via a predetermined breaking point B with optional use of a linker C and/or spacer D.

In the following, sites are shown which are suitable sites on E for derivatisation to give perfluoroderivatives: a) perfluorocarbyl derivatisation of nucleobases, nucleosides, nucleotides, b) perfluorocarbyl derivatisation of peptide nucleic acid monomers and oligomers, peptide nucleic acids, c) perfluorocarbyl derivatisation of oligonucleotides.

In a first variant, the perfluorocarbyl derivatisation of nucleobases used to make E can occur at all accessible places in the molecule, with NH₂ and NH groups being preferred perfluorocarbyl derivatisation sites (see Diagram 2). The only limitation occurs during the conversion to the nucleoside at the respective NH group (arrow); see diagram 2.

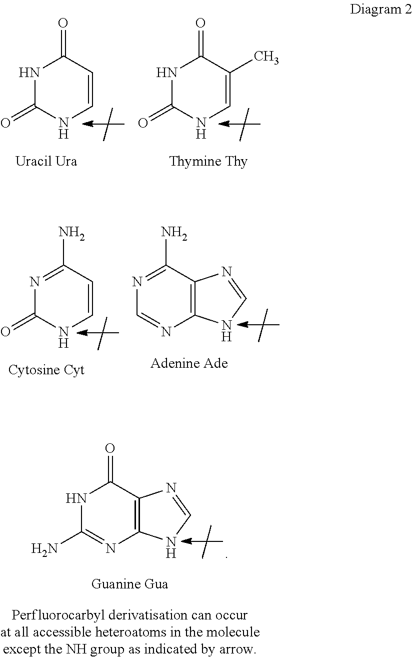

In a second variant, a perfluorocarbyl derivatisation is also possible at sites in the sugar molecule of nucleosides, with preferred sites being the 2′, 3′ and/or 5′ position of the ribose (see Diagram 3). The 2′ position of the ribose can also be modified in the form of —NH₂ and/or —SH, the preferred perfluorocarbyl derivatisation sites corresponding to 2′-NH₂, 2′-SH, etc.

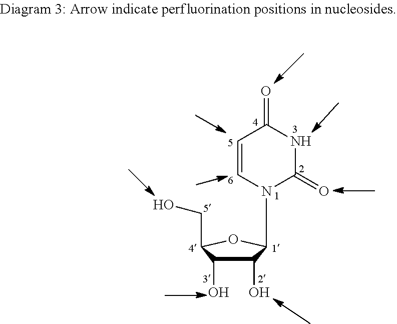

In addition to the perfluorocarbyl derivatisation sites at the ribose in 2′ and 3′ positions, according to a third variant, there are additional perfluorocarbyl derivatisation sites at the phosphate group of nucleotides, such as at the free OH group or on at least one oxygen atom; see diagram 4.

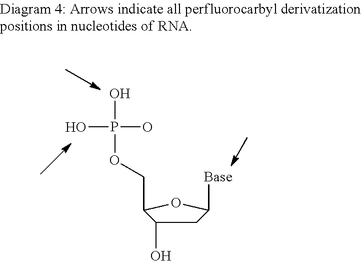

For DNA molecules, the same perfluorocarbyl derivatization positions are possible. Arrows indicate positions within the molecule which can be modified, either by adding a fluorocarbyl group or by replacing an indicated substituent by a fluorocarbyl group whilst respecting the normal rules of valency.

Alternatively, however, perfluorocarbyl derivatisation sites can also be realized on modified sugar-phosphate structures with heteroatoms such as, for example, S or N or others, as shown in diagram 5.

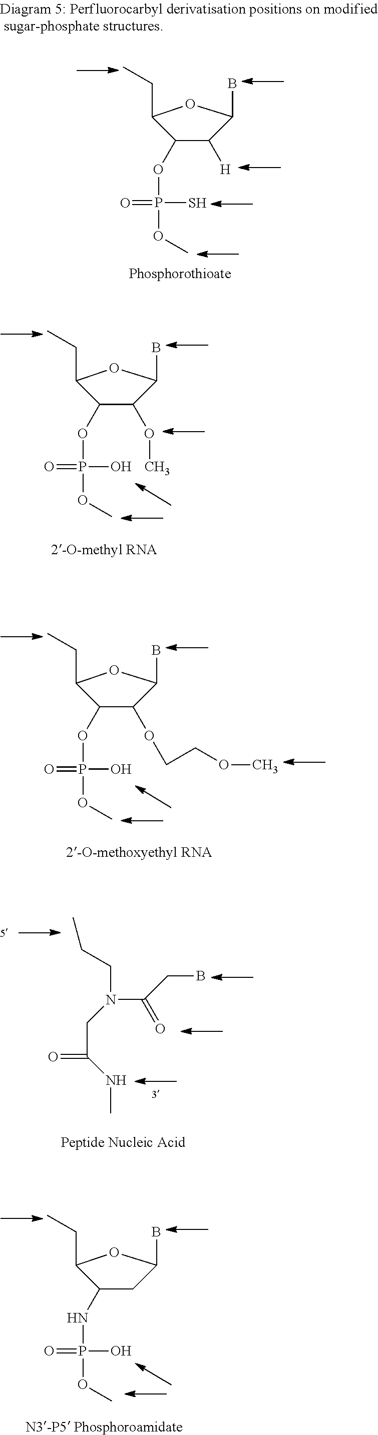

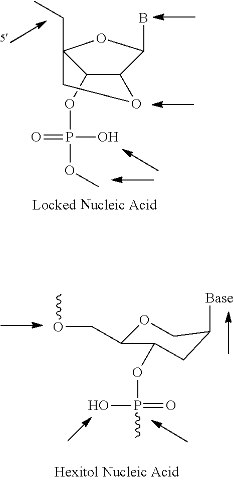

Arrows indicate positions within the molecule which can be modified, either by adding a fluorocarbyl group or replacing an indicated substituent by a fluorocarbyl group, whilst respecting the normal rules of valency.

In a fourth variant, the perfluorocarbyl derivatisation of peptide nucleic acid monomers, peptide nucleic acid oligomers and peptide nucleic acids can occur. These molecules are analogs of nucleic acids. The sugar-phosphate backbone is replaced by a pseudopeptide, for example by aminoethylglycine units that are joined together by neutral amide bonds. They are very stable, as they cannot be broken down either by nucleases or by proteases. They hybridize more stringently with complementary DNA and RNA sequences as the original oligomers, and additional perfluorocarbyl derivatisation sites exist on NH₂ and carboxy groups and on the O-atom, as well as on functional groups of modified peptide structures; see diagram 6.

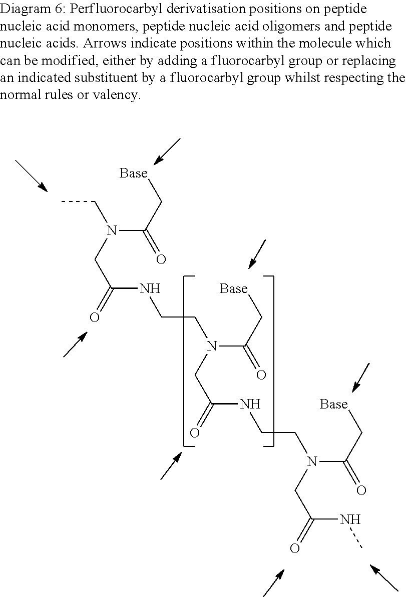

In a fifth variant, the perfluorocarbyl derivatisation of nucleic acid oligomers or oligonucleotides and of nucleic acid macromolecules can occur, with different approaches being possible.

In a first approach, perfluorocarbyl derivatives of nucleotides are built directly into the desired RNA or DNA sequence, for example by using solid-phase synthesis or PCR, being preferred to use perfluorocarbyl nucleotide derivatives that are perfluorocarbylated at the 2′ position (i.e., in the 2′ position of the ribose). The reason for this is that, firstly, perfluorocarbyl derivatisation at this site does not lead to chain breakage during polymerization or synthesis and, secondly, the interactions between base pairs are not disturbed; see the two upper examples of diagram 7.

While the 2′ OH position in RNA molecules can easily be derivatised with perfluorocarbyl residues, in DNA molecules the 2′H position must first be functionalized, for example by reactive groups such as NH₂ instead of H or 2′-amino-2′-deoxyuridine.

In a second approach, the perfluorocarbyl derivatives of RNA nucleotides are incorporated into the DNA sequence or perfluorocarbyl derivatives of DNA nucleotides are synthesized on the 5′ or 3′ ends of oligonucleotides or of DNA macromolecules; see third example in diagram 7 (see below). In this approach, accordingly, perfluorocarbyl derivatives such as perfluorocarbyl derivatised nucleotides are bound to the ends of the finished oligonucleotide preferably by means of chemical synthesis. The nucleotides used can be derivatized with perfluorocarbyl groups at any possible position as described above, with 2′- and 5′-perfluorocarbyl nucleotides being preferred. The only exception here is a nucleotide perfluorocarbylated at the 5′ position with C₈F₁₇.

According to the present invention, the compounds can be present in different constructs with different structures, importantly the compounds can have the following additional basic structures depending on the presence of the C, D, F and G.

-A-B-E-F-G, (III)

-A-B-D-B-E-F-G, (IV)

-A-B-C-B-E-F-G, (V)

-A-B-E, (VI)

-A-B-D-B-E, (VII)

-A-B-C-B-E, (VIII)

-A-B-E-F, (IX)

-A-B-D-B-E-F, (X)

-A-B-C-B-E-F, (XI)

-A-B-E-G, (XII)

-A-B-D-B-E-G, (XIII)

-A-B-C-B-E-G, (XIV)

-A-B-E-A′, (XV)

-A-B-D-B-E, (XVI)

-A-B-C(F′)-B-E, (XVII)

-A-B-C(G′)-B-E, (XVIII)

-A-B-C(F′, G′)-B-E, (XIX)

-A-B-C(F′)-E, (XX)

-A-B-C(G′)-E, (XXI)

-A-B-C(F′, G′)-E, (XXII)

For examples, reference is made to the following compounds:

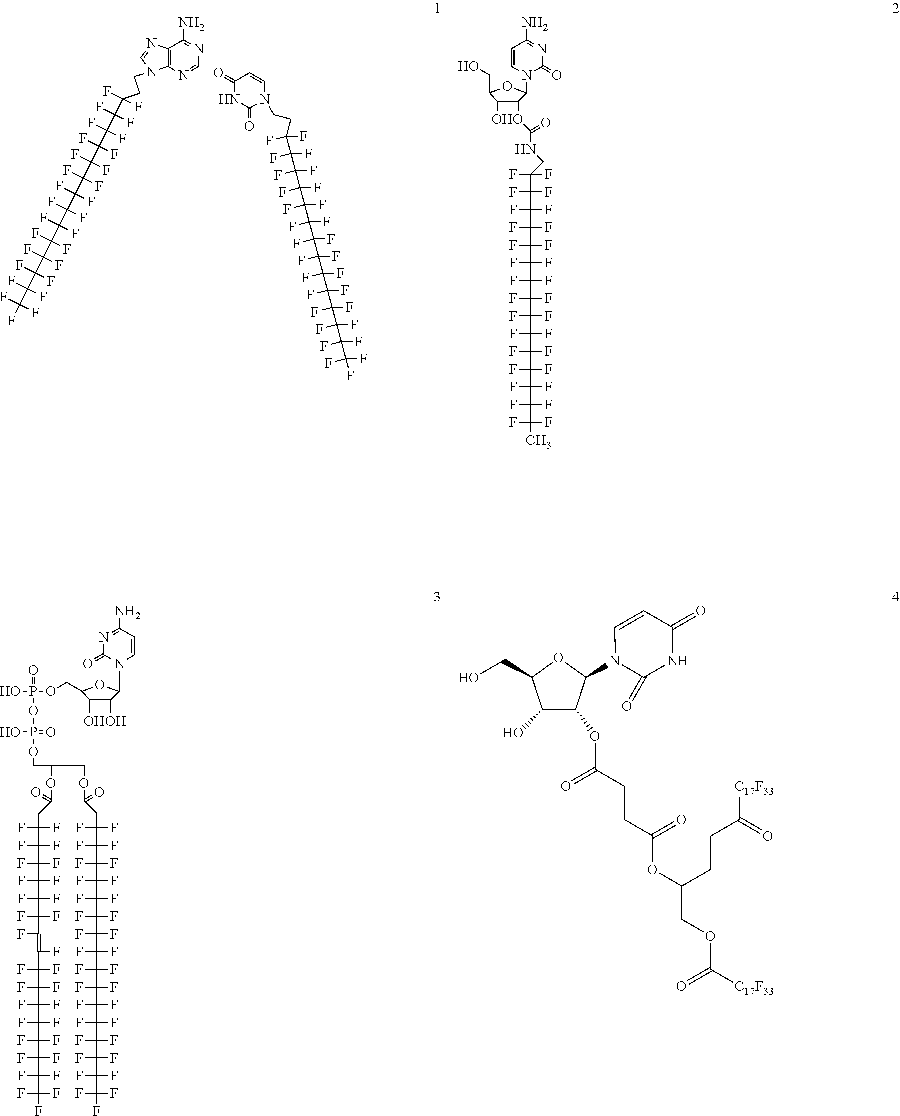

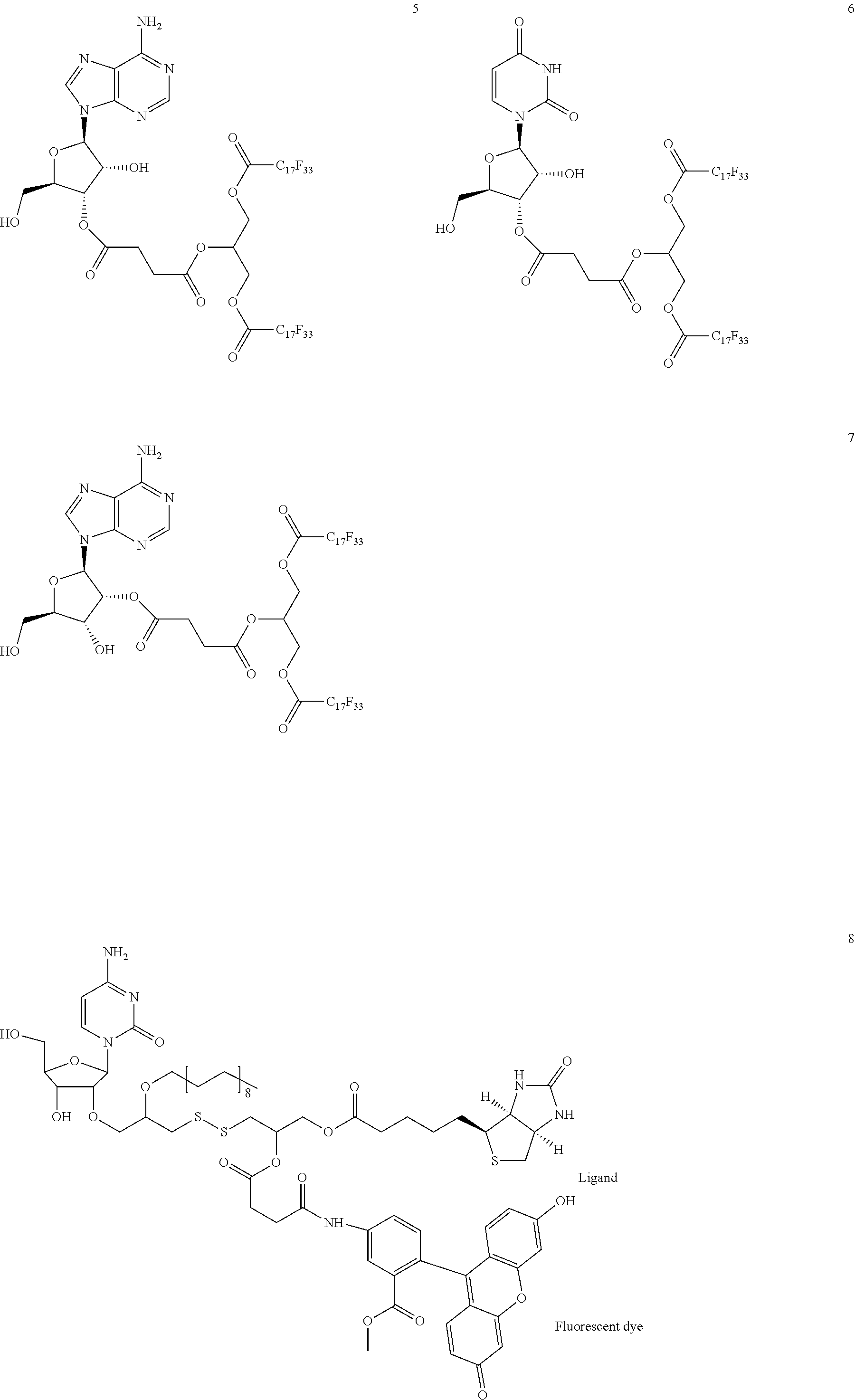

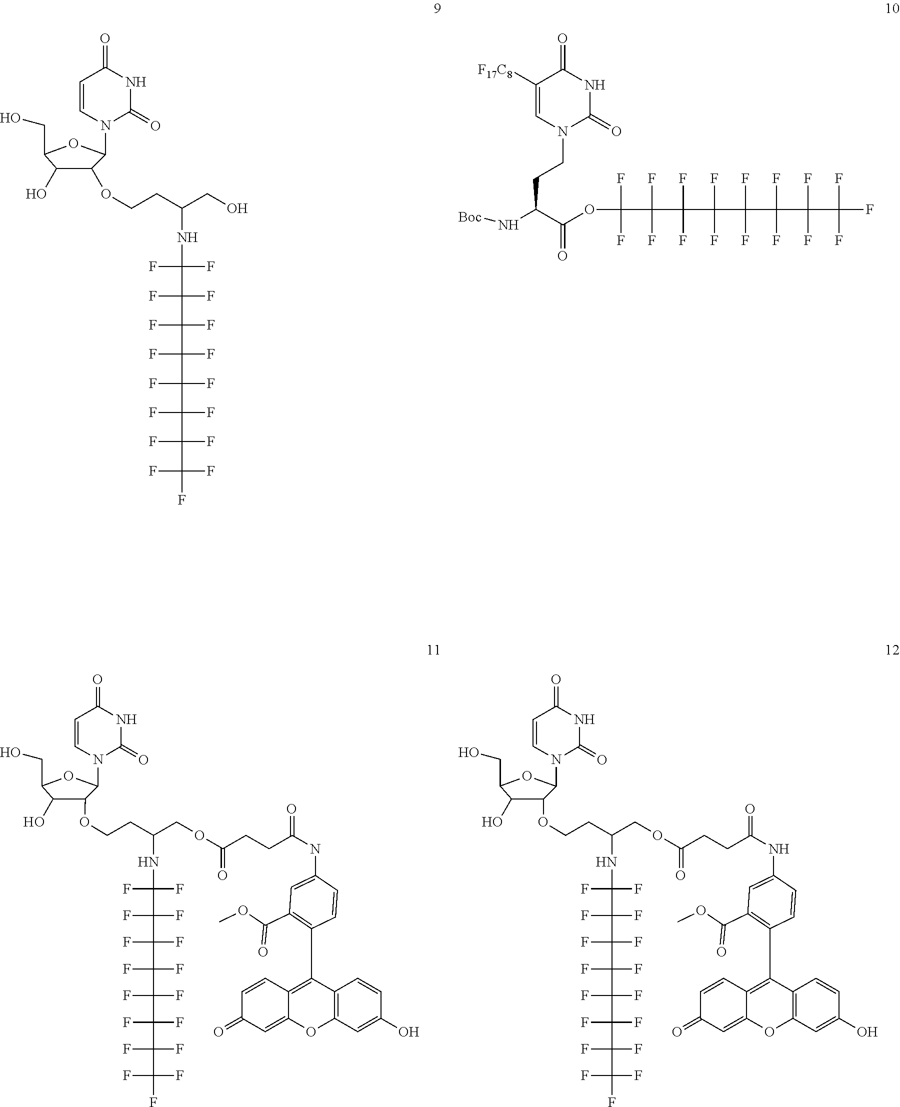

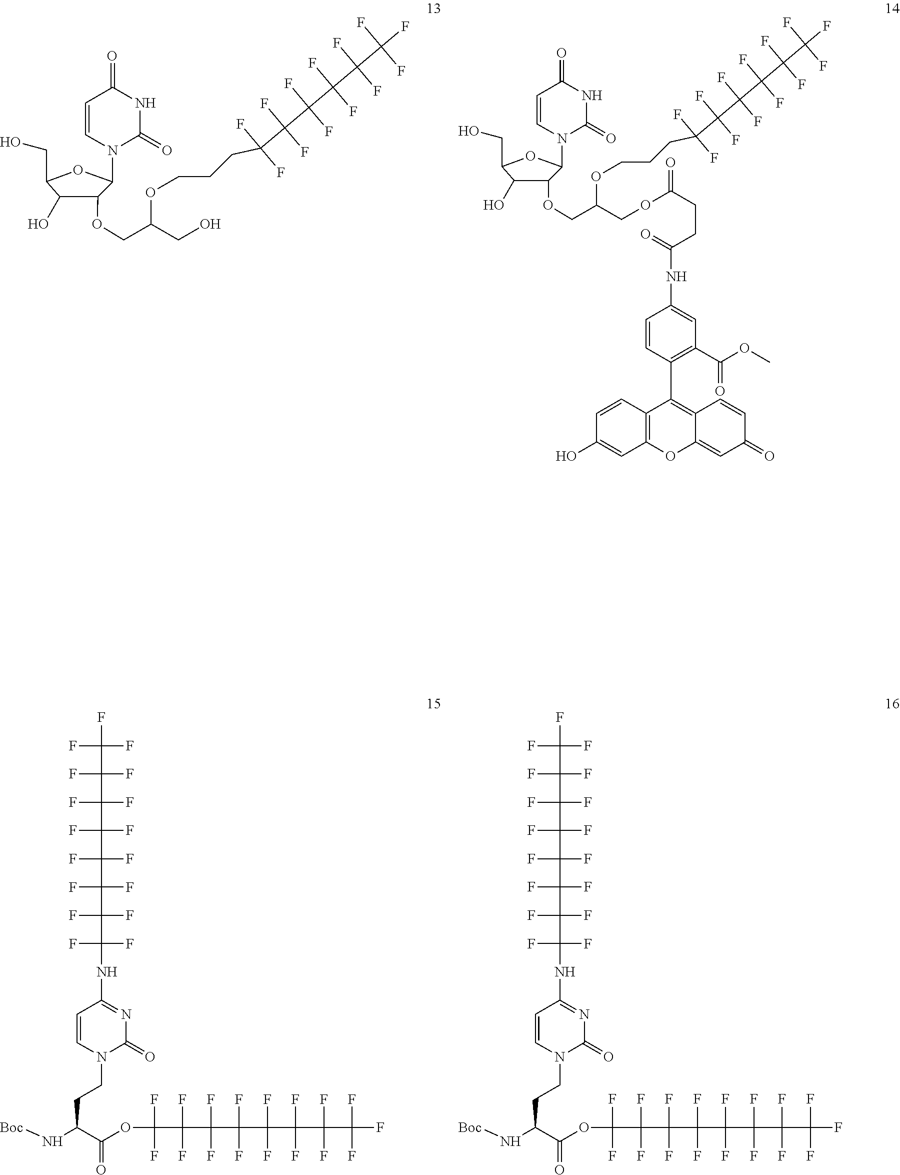

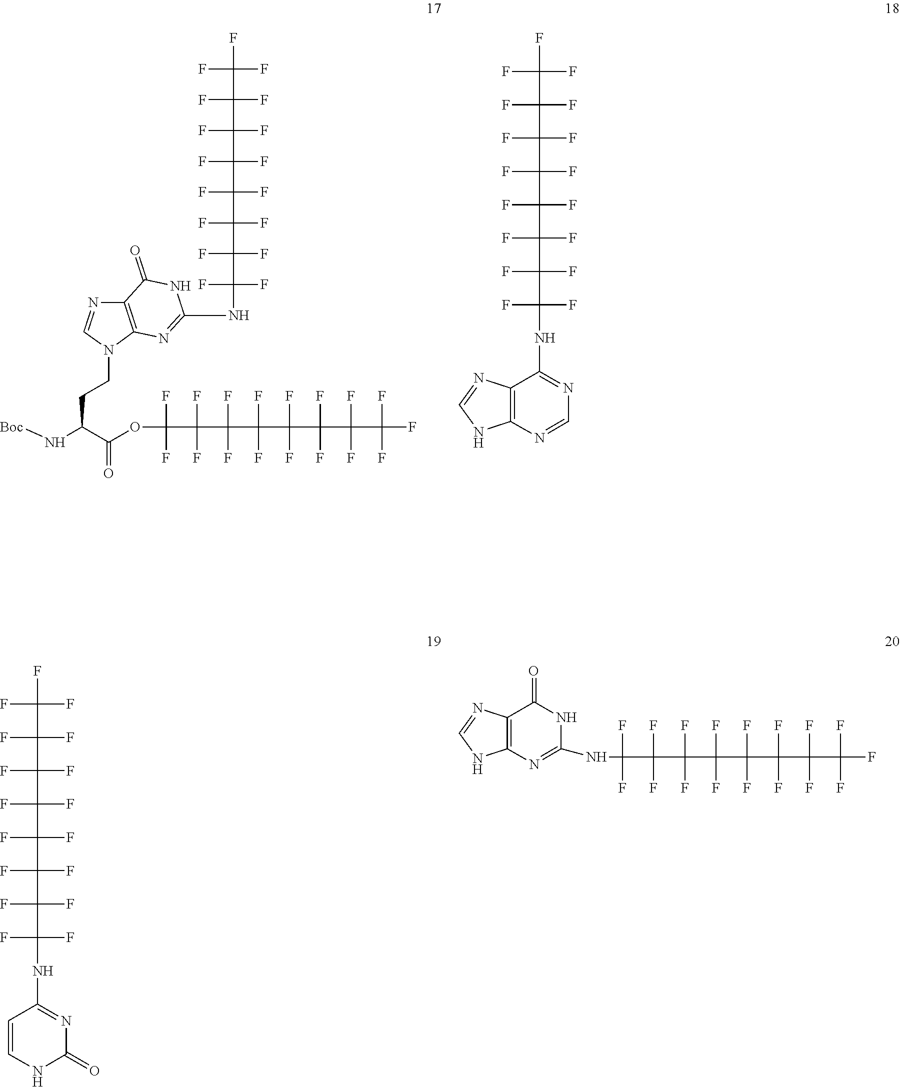 [SMILES: CC(C)(C)OC(=O)N[C@@H](CCn1cnc2c(=O)[nH]c(NC(F)(F)C(F)(F)C(F)(F)C(F)(F)C(F)(F)C(F)(F)C(F)(F)C(F)(F)F)nc21)C(=O)OC(F)(F)C(F)(F)C(F)(F)C(F)(F)C(F)(F)C(F)(F)C(F)(F)C(F)(F)F ; FC(F)(F)C(F)(F)C(F)(F)C(F)(F)C(F)(F)C(F)(F)C(F)(F)C(F)(F)Nc1ncnc2[nH]cnc12 ; O=c1nc(NC(F)(F)C(F)(F)C(F)(F)C(F)(F)C(F)(F)C(F)(F)C(F)(F)C(F)(F)F)cc[nH]1 ; O=c1[nH]c(NC(F)(F)C(F)(F)C(F)(F)C(F)(F)C(F)(F)C(F)(F)C(F)(F)C(F)(F)F)nc2[nH]cnc12]

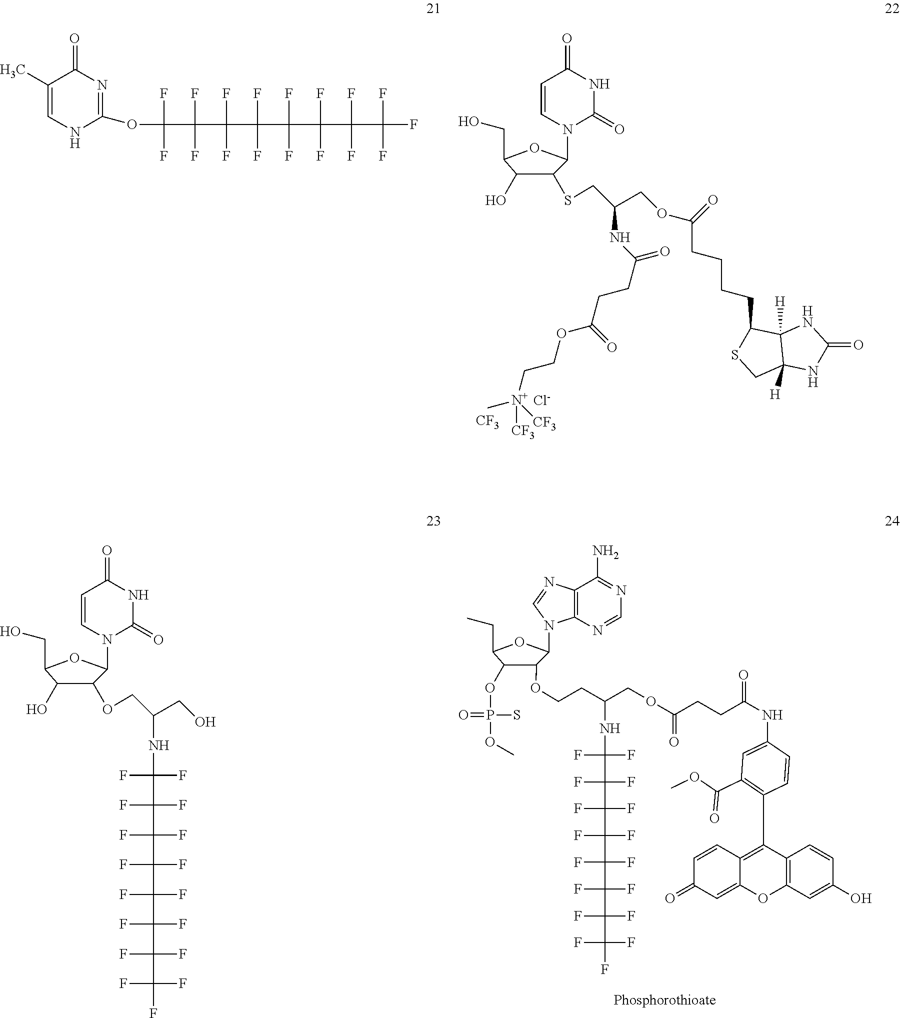

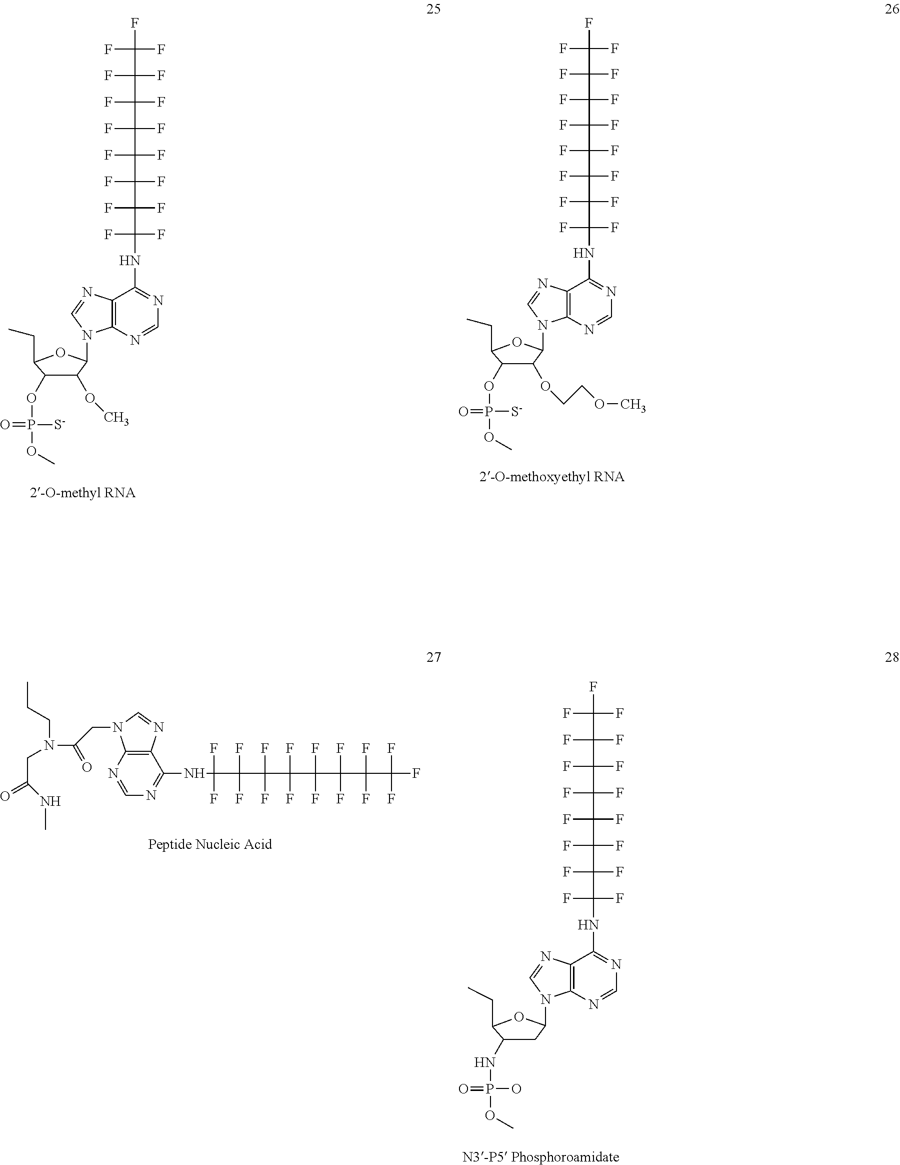 [SMILES: CCC1OC(n2cnc3c(NC(F)(F)C(F)(F)C(F)(F)C(F)(F)C(F)(F)C(F)(F)C(F)(F)C(F)(F)F)ncnc32)C(OC)C1OP(=O)([S-])OC ; CCC1OC(n2cnc3c(NC(F)(F)C(F)(F)C(F)(F)C(F)(F)C(F)(F)C(F)(F)C(F)(F)C(F)(F)F)ncnc32)C(OCCOC)C1OP(=O)([S-])OC ; CCCN(CC(=O)NC)C(=O)Cn1cnc2c(NC(F)(F)C(F)(F)C(F)(F)C(F)(F)C(F)(F)C(F)(F)C(F)(F)C(F)(F)F)ncnc21 ; CCC1OC(n2cnc3c(NC(F)(F)C(F)(F)C(F)(F)C(F)(F)C(F)(F)C(F)(F)C(F)(F)C(F)(F)F)ncnc32)CC1NP(=O)(O)OC]

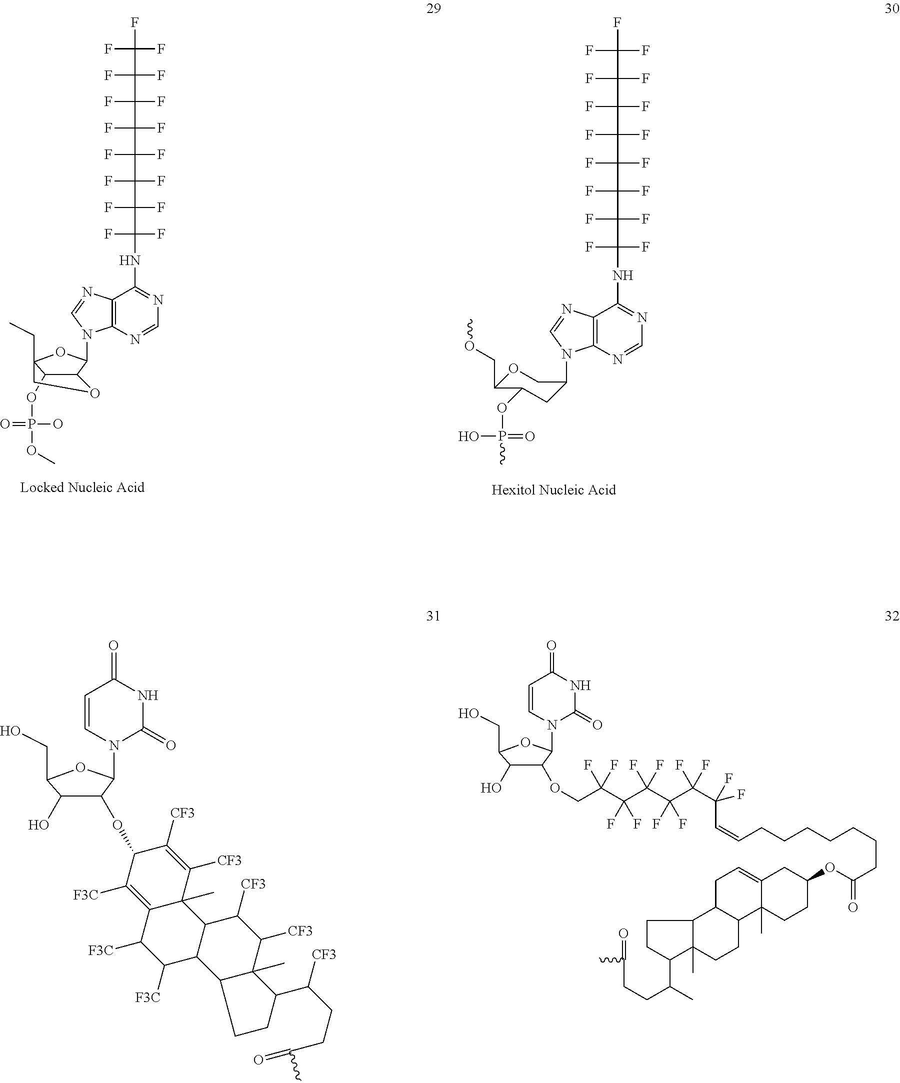

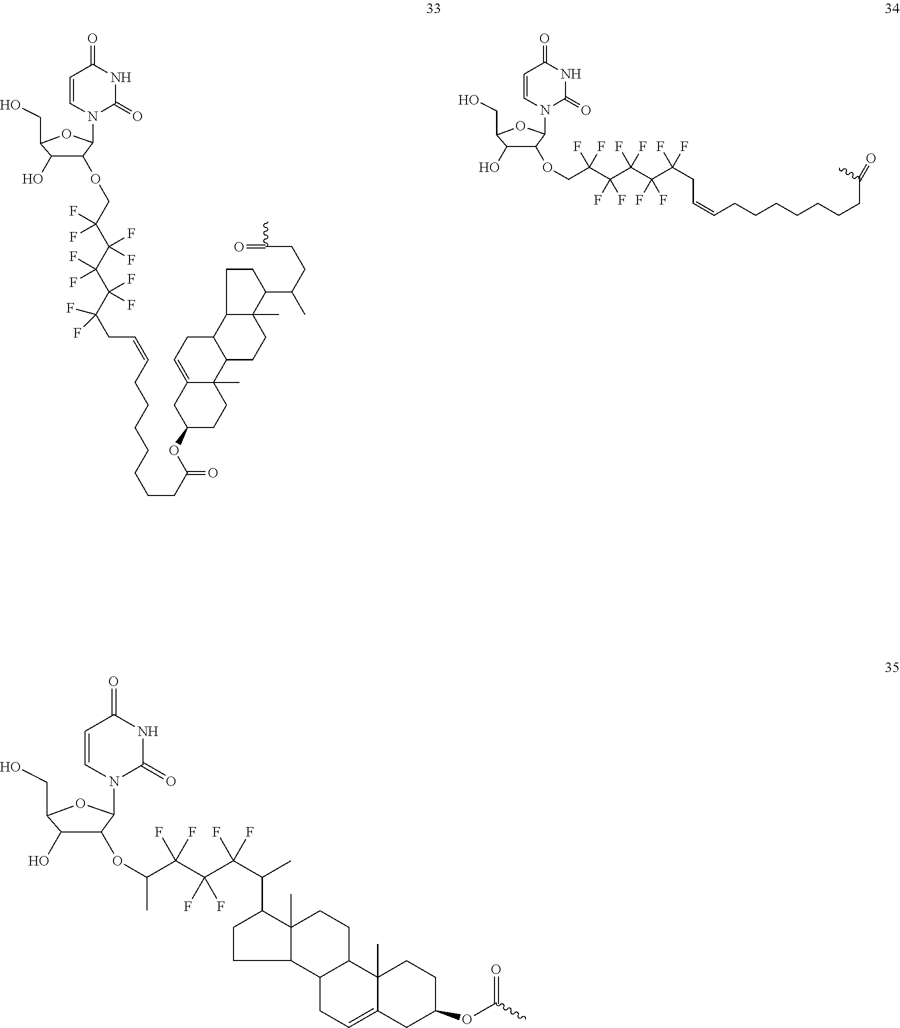

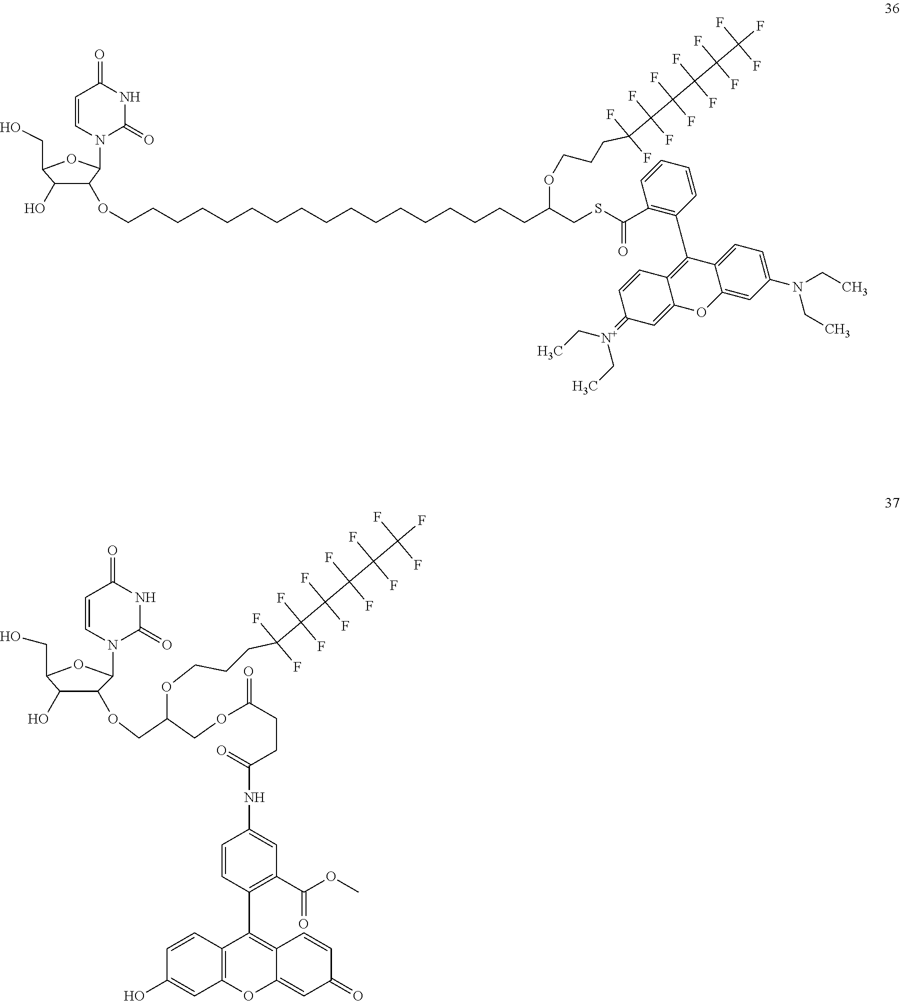 [SMILES: CCN(CC)c1ccc2c(-c3ccccc3C(=O)SCC(CCCCCCCCCCCCCCCCCOC3C(O)C(CO)OC3n3ccc(=O)[nH]c3=O)OCCCC(F)(F)C(F)(F)C(F)(F)C(F)(F)C(F)(F)C(F)(F)F)c3ccc(=[N+](CC)CC)cc-3oc2c1 ; COC(=O)c1cc(NC(=O)CCC(=O)OCC(COC2C(O)C(CO)OC2n2ccc(=O)[nH]c2=O)OCCCC(F)(F)C(F)(F)C(F)(F)C(F)(F)C(F)(F)C(F)(F)F)ccc1-c1c2ccc(=O)cc-2oc2cc(O)ccc12]

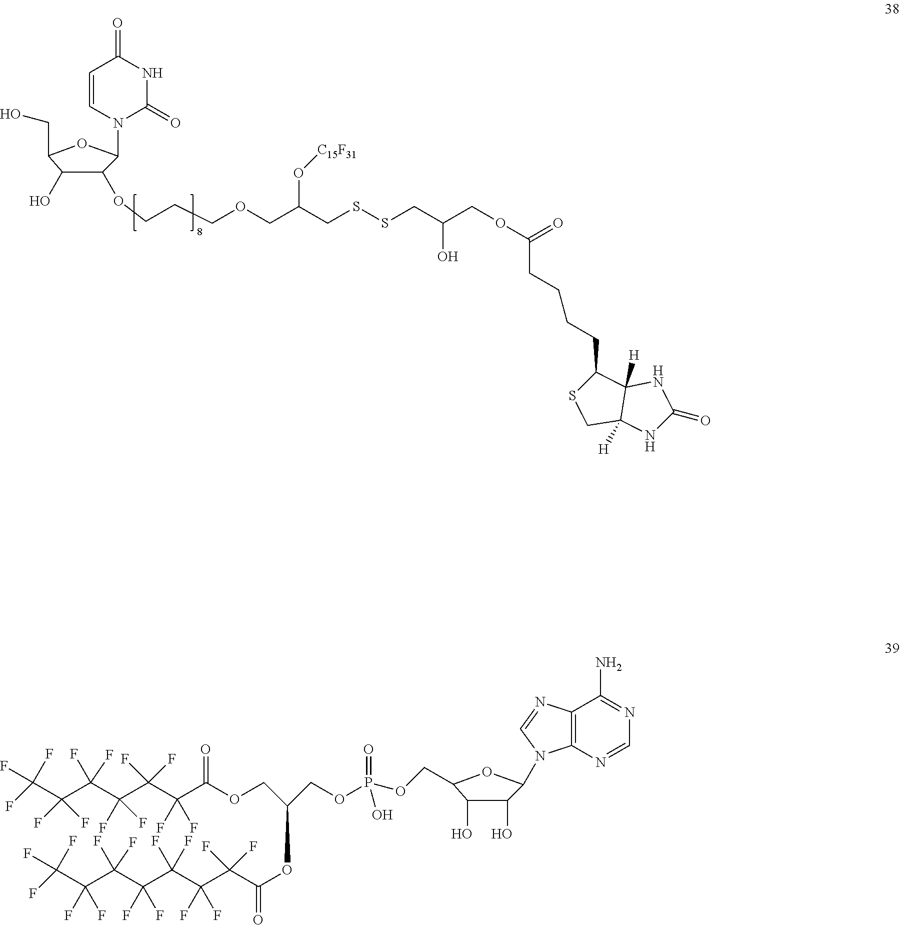

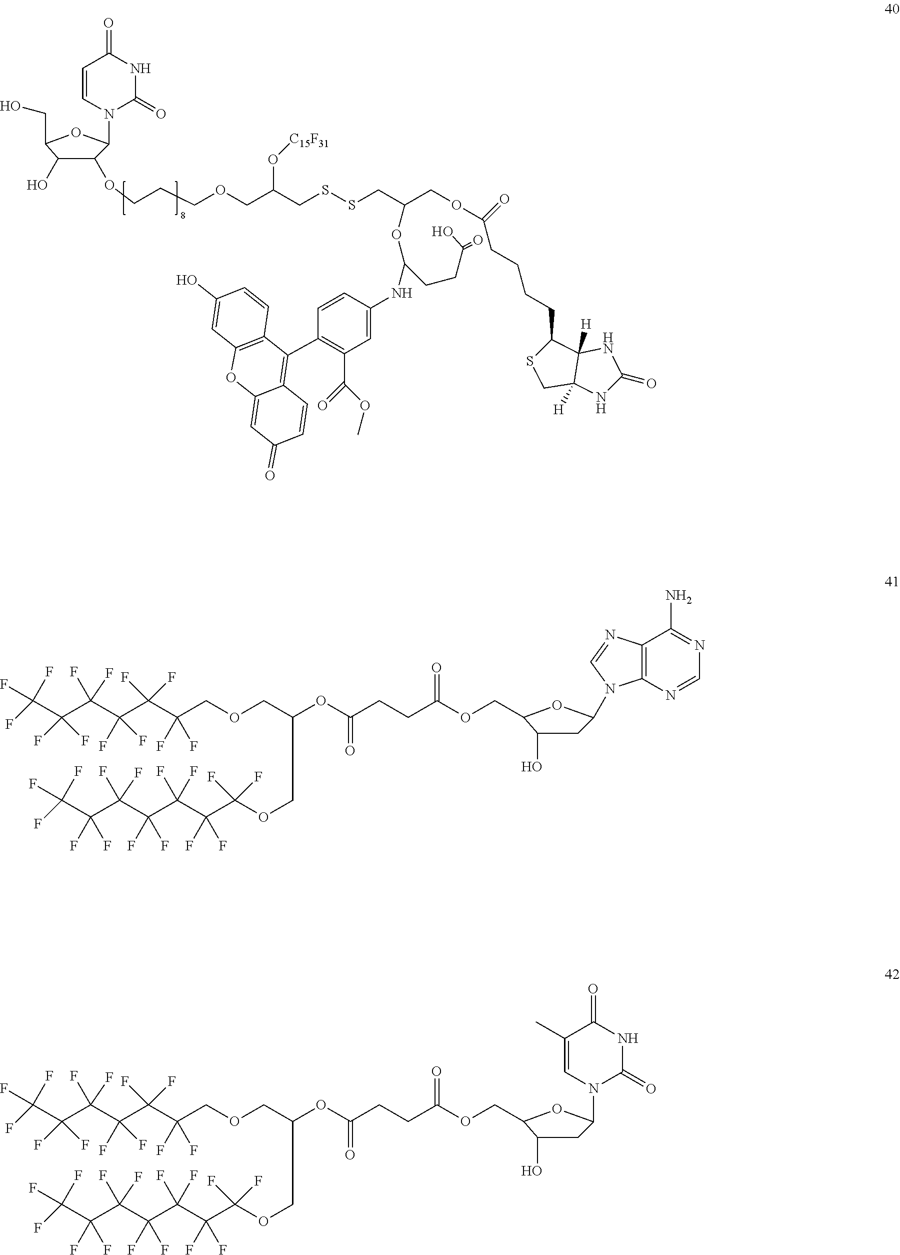

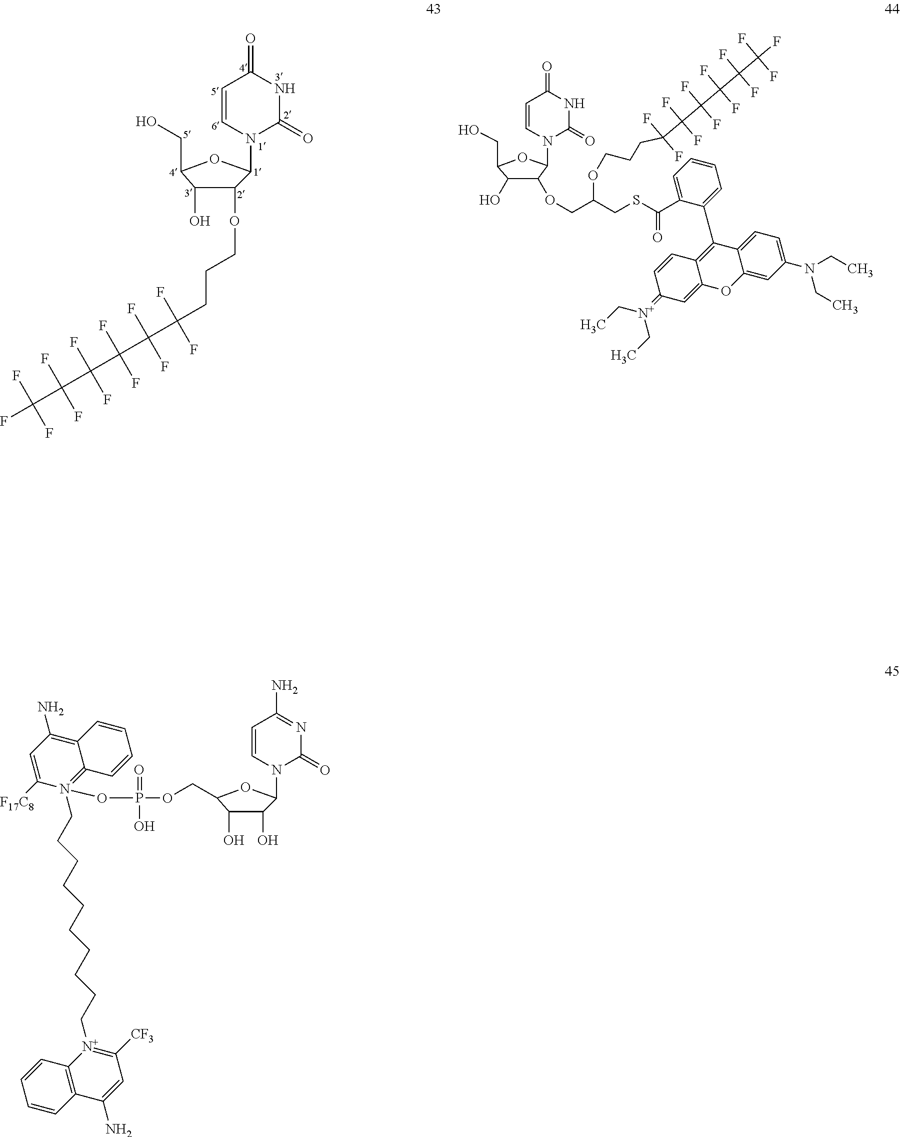

Wavy lines or dashed lines in the structures in structures 24-35 indicate positions where further (the same or different) structures of the present invention can be bound.

The compounds of the present method are synthesized by known chemical methods which enable a successive linking of the individual molecule building blocks, for example by means of addition, substitution, condensation, etherification or esterification. Such synthesis paths are known to a synthetic chemist as a person skilled in the art.

### BRIEF DESCRIPTION OF THE DRAWINGS

For a better understanding of the present invention, the invention is explained in the following on the basis of sample embodiments with reference to the figures without being limited to these examples.

FIG. 1 shows microscopic images of cells transfected with the compounds according to the invention, and

FIG. 2 shows a FACS analysis of cells transfected with the compounds according to the invention.

### Sample Embodiment 1: Synthesis of Perfluorocarbyl Nucleobases

The perfluorocarbylation of nucleobases can be carried out by means of Williamson's ether synthesis. The NH₂ groups are relatively easily accessible. Thymine does not have an NH₂ group but can occur in 6 tautomeric structures, of which 4 structures have an OH group susceptible to perfluorocarbylation.

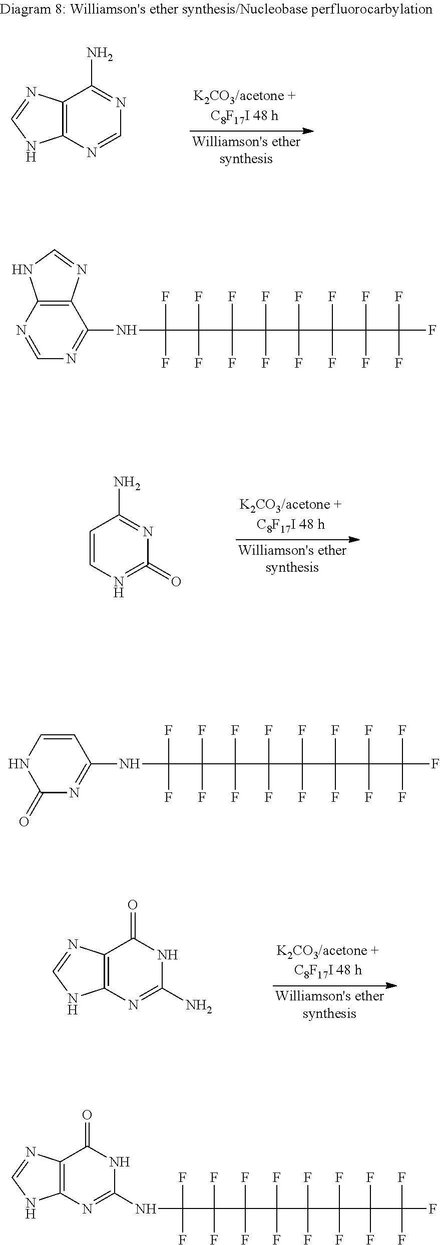

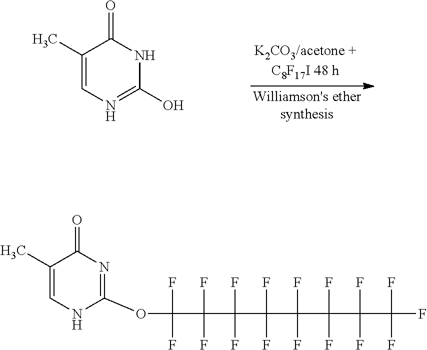

### Sample Embodiment 2: Synthesis of 2′-Perfluorocarbyl Nucleosides on the Basis of 2′-Perfluorocarbyl Uridine

Uridine is an important component of RNA. The incorporation of uridine into RNA chain occurs via the OH groups of the 3′ and 5′ position in the sugar. Therefore, the 2′ position of uridine is especially suitable for the introduction of substituents without adversely affecting the bonding sites of the nucleoside. Various 2′-substituted uridines are known in which the linking occurs via 2′ ethers or esters, 2′ thioethers or esters, 2′ acid amides or 2′ carbamates or even via 2-C; see diagram 9.

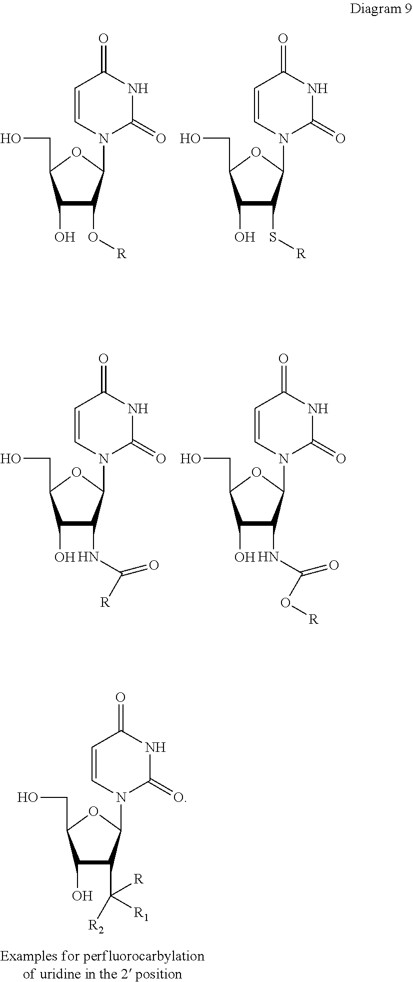

For the synthesis of uridines with perfluoroalkyl in the 2′ position, new syntheses have been worked out. For instance, the direct linking to the 2′-OH group is carried out via an ether function and an ester function; see diagram 10.

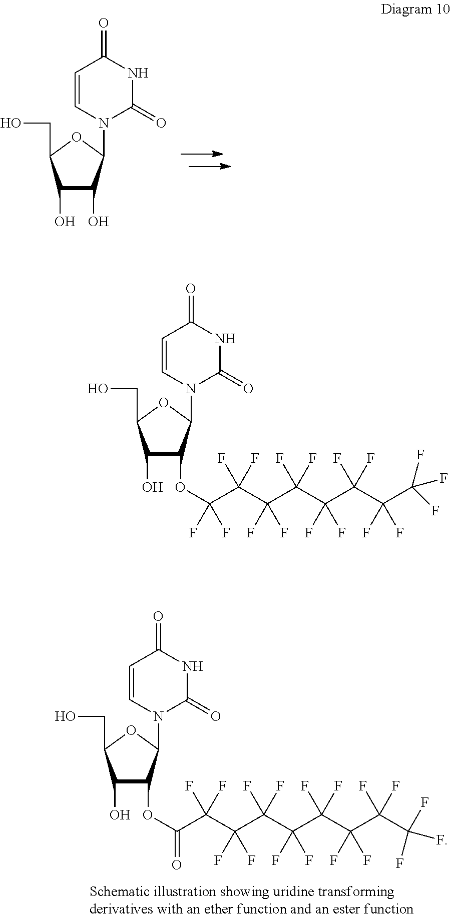

Another possibility is the linking of the perfluorocarbylated alkyl group to the 2′-OH group via a spacer and a predetermined breaking point; see diagram 11.

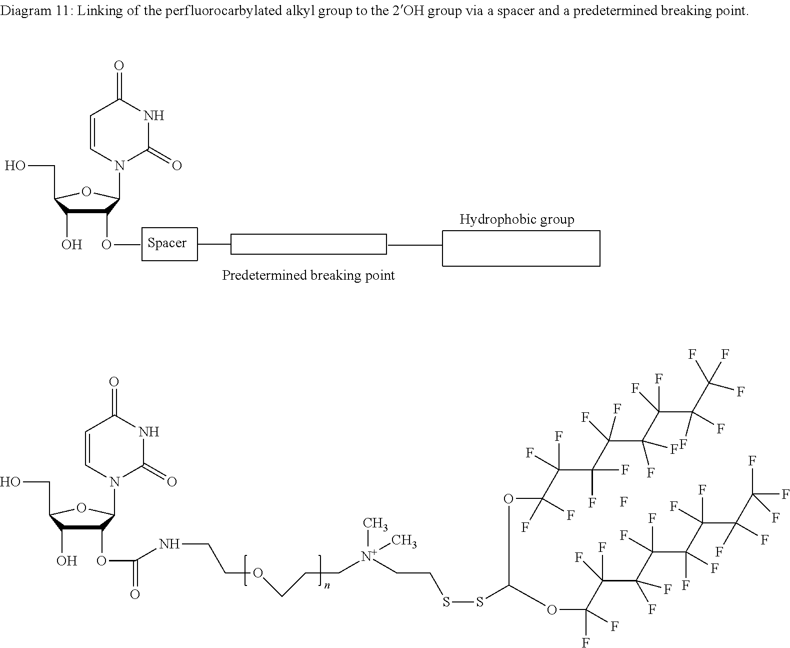

For all the reactions depicted in diagrams 9 to 11, the 3′ and 5′-OH groups have to be protected. Silyl protective groups are suitable for this purpose. However, it must be noted here that an equilibrium between the 2′- and 3 ‘-substituted uridine can occur in some cases (by acyl group migration). For this reason, the linking of the hydrophobic group to the 2’ position of the uridine was done via an ether function or via an amino function (2′-amino-2′-deoxyuridine).

### Sample Embodiment 3

Synthesis of 2′-perfluorocarbylated nucleosides under protection of 3 ‘and 5′OH groups followed by perfluoroalkylation of the 2′OH group Perfluorocarbylated hydrophobic groups are bound to the 2’-OH group via an ether link. For this, in the first step, the OH groups were protected in 3′ and 5′ positions using Dichloro-tetraisopropyldisiloxane. In the next step, the OH group was subsequently etherified in the 2′ position with 1-iodoperfluorooctane or 1-iodoperfluoroundecane and deprotected. Depending on the course of reaction, the intermediate and end products of the reaction were purified by means of preparative chromatography. The reactions were carried out under the exclusion of moisture and under inert gas (argon). The solvents used had to be dried before being used, too.

Specifically, one reacts a perfluorohydrocarbyl halide (C₈F₁₇Br or C₈F₁₇I) and a protected uridine (with the 3′-OH and 5′-OH groups on the uridine first protected by reaction with 3′,5′-diethylbutylsiloxanyl). The reaction with the perfluorohydrocarbyl halide takes place at the 2′-OH group of the uridine. For this purpose, C₈F₁₇Br (or C₈F₁₇I) is bound to the 2′-OH group of the uridine using Williamson's ether synthesis, thus yielding uridine-2′0-C₈F₁₇ (with 3′,5′-diethylbutylsiloxanyl). For this purpose, the reaction is carried out according to Monokanen et al. 1991 and Monokanen et al. 1993 thus yielding protected Uridine-2′0-C₈F₁₇ (with 3′,5′-diethylbutylsiloxanyl). Finally, the protective groups are split off; see diagram 12.

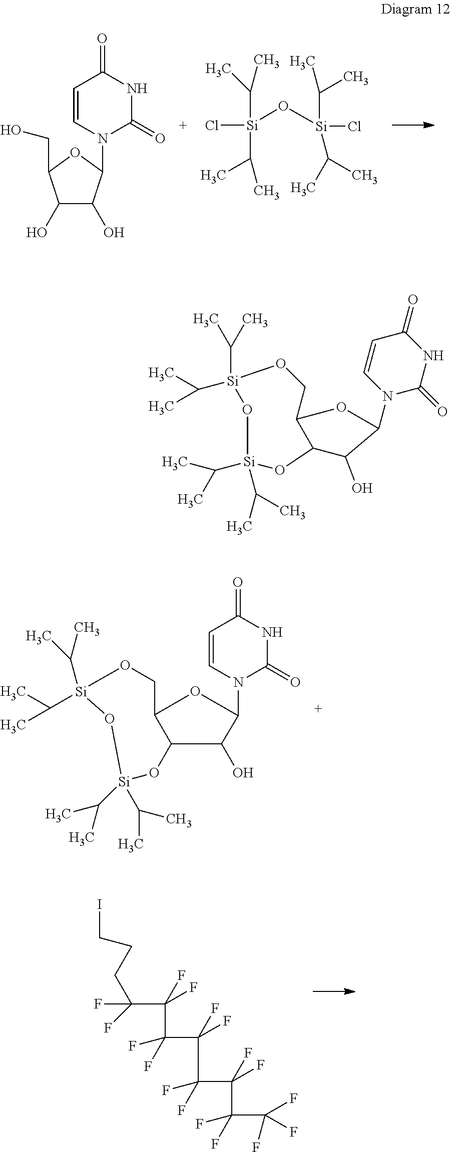 [SMILES: O=c1ccn(C2OC(CO)C(O)C2O)c(=O)[nH]1 ; CC(C)[Si](Cl)(O[Si](Cl)(C(C)C)C(C)C)C(C)C ; CC(C)[Si]1(C(C)C)OCC2OC(n3ccc(=O)[nH]c3=O)C(O)C2O[Si](C(C)C)(C(C)C)O1 ; CC(C)[Si]1(C(C)C)OCC2OC(n3ccc(=O)[nH]c3=O)C(O)C2O[Si](C(C)C)(C(C)C)O1 ; FC(F)(F)C(F)(F)C(F)(F)C(F)(F)C(F)(F)C(F)(F)C(F)(F)C(F)(F)CCCI]

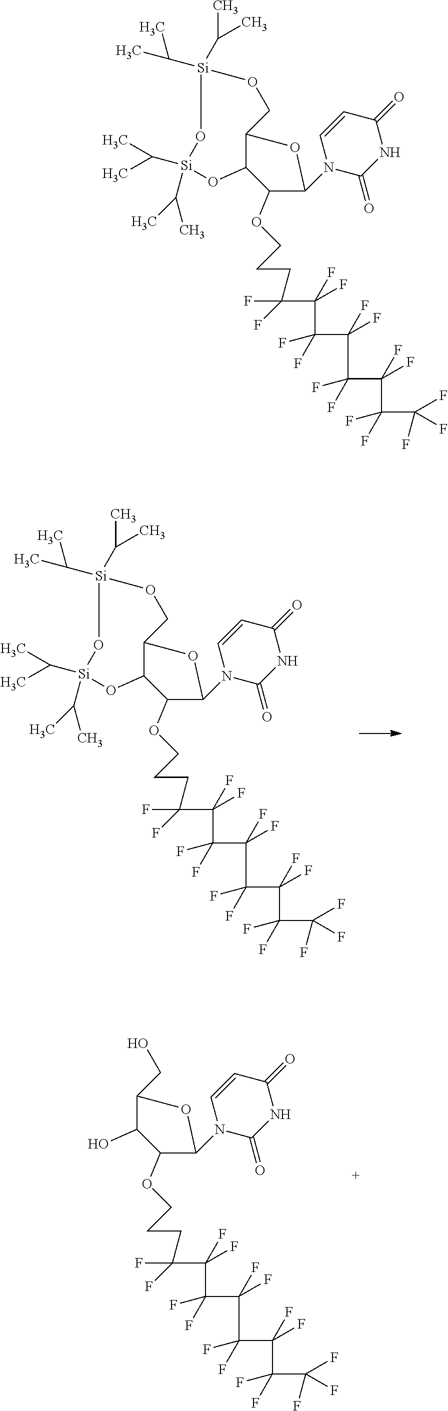 [SMILES: CC(C)[Si]1(C(C)C)OCC2OC(n3ccc(=O)[nH]c3=O)C(OCCCC(F)(F)C(F)(F)C(F)(F)C(F)(F)C(F)(F)C(F)(F)C(F)(F)C(F)(F)F)C2O[Si](C(C)C)(C(C)C)O1 ; CC(C)[Si]1(C(C)C)OCC2OC(n3ccc(=O)[nH]c3=O)C(OCCCC(F)(F)C(F)(F)C(F)(F)C(F)(F)C(F)(F)C(F)(F)C(F)(F)C(F)(F)F)C2O[Si](C(C)C)(C(C)C)O1 ; O=c1ccn(C2OC(CO)C(O)C2OCCCC(F)(F)C(F)(F)C(F)(F)C(F)(F)C(F)(F)C(F)(F)C(F)(F)C(F)(F)F)c(=O)[nH]1]

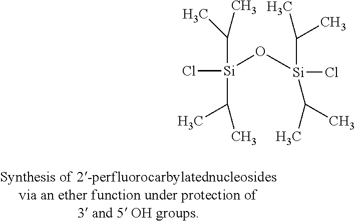 [SMILES: CC(C)[Si](Cl)(O[Si](Cl)(C(C)C)C(C)C)C(C)C]

### Sample Embodiment 4: Perfluoroacylation via a 2′-Amino Function of 2′-Amino-2′-Deoxyuridine

The synthesis starts with 2′-amino-2′-deoxyuridine, which is commercially available. This was converted with perfluorocarboxylic acids into acid amide (see Diagram 13). Here inert gas was used under the exclusion of moisture, too. The end product was purified using preparative column chromatography. The primary OH group in the 5′ position of the uridine was protected with DMT. For the analogous reactions at other positions of the nucleic acid components, adequate protective groups can be added or omitted.

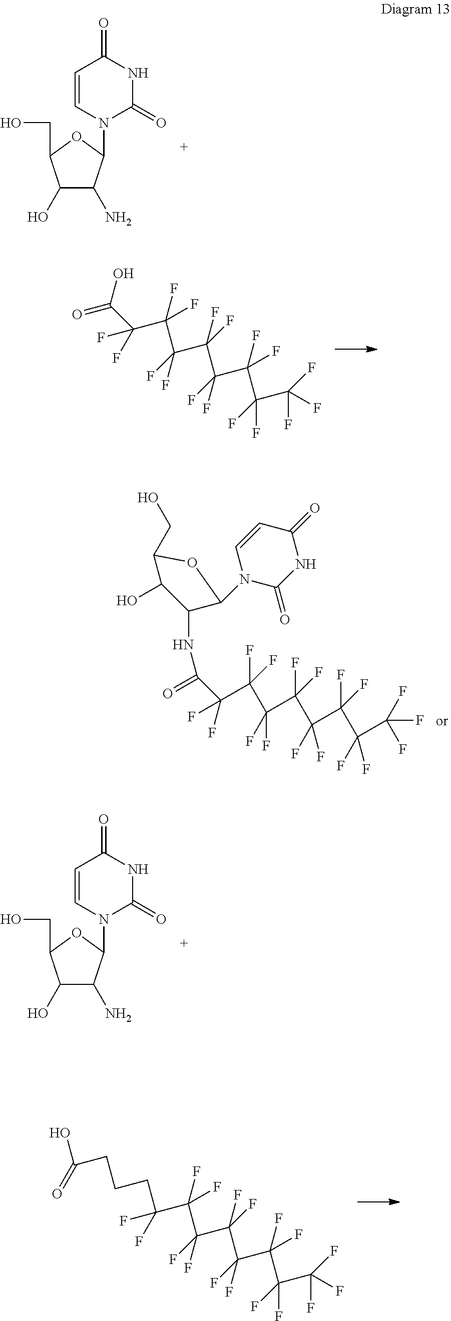 [SMILES: NC1C(O)C(CO)OC1n1ccc(=O)[nH]c1=O ; O=C(O)C(F)(F)C(F)(F)C(F)(F)C(F)(F)C(F)(F)C(F)(F)C(F)(F)C(F)(F)F ; O=C(NC1C(O)C(CO)OC1n1ccc(=O)[nH]c1=O)C(F)(F)C(F)(F)C(F)(F)C(F)(F)C(F)(F)C(F)(F)C(F)(F)C(F)(F)F ; NC1C(O)C(CO)OC1n1ccc(=O)[nH]c1=O ; O=C(O)CCCC(F)(F)C(F)(F)C(F)(F)C(F)(F)C(F)(F)C(F)(F)C(F)(F)C(F)(F)F]

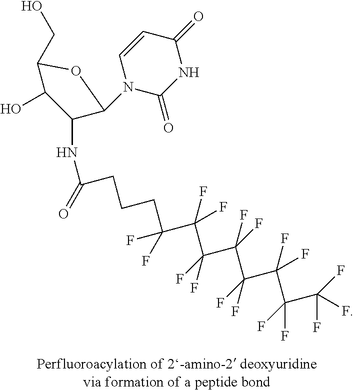 [SMILES: O=C(CCCC(F)(F)C(F)(F)C(F)(F)C(F)(F)C(F)(F)C(F)(F)C(F)(F)C(F)(F)F)NC1C(O)C(CO)OC1n1ccc(=O)[nH]c1=O]

### Sample Embodiment 5: Synthesis of a Perfluoroalkylated Nucleoside with Biotin as a Recognition Sequence

The starting point of the synthesis was a 2′-modified uridine nucleoside with an amino alcohol side chain. The amino alcohol side chain was perfluoroalkylated at its amino function with C₈H₁₇OH. Using 1-ethyl-3 -(3 -dimethylaminopropyl)carbodiimide (EDC), hydroxybenzotriazole (HOBt), and diisopropylethylamine (DIPEA) chemoselective alkylation was achieved without an attack on the alcoholic hydroxyl group. The remaining primary hydroxyl function was then esterified with biotin.

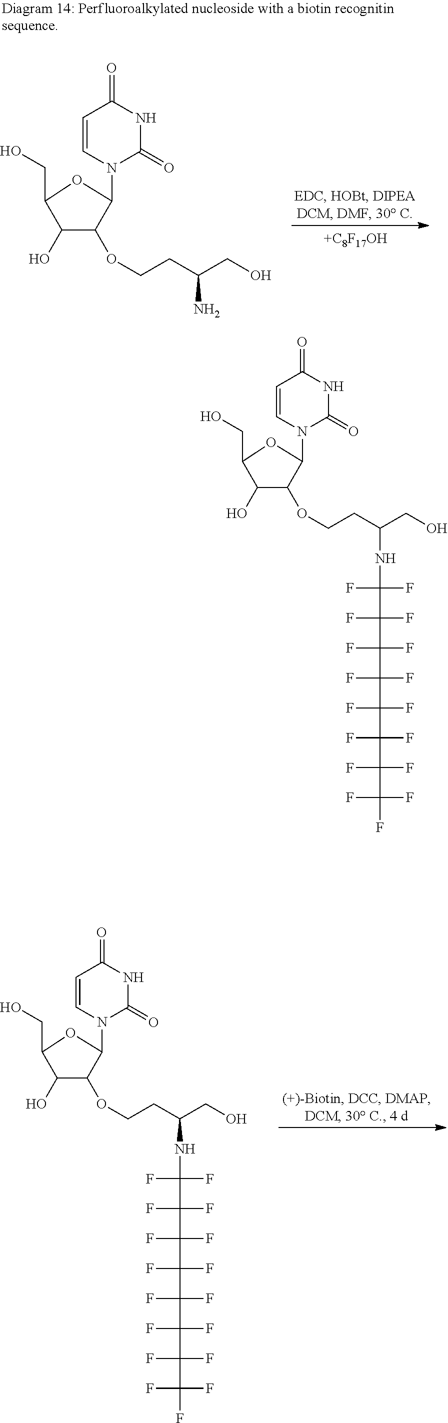 [SMILES: N[C@H](CO)CCOC1C(O)C(CO)OC1n1ccc(=O)[nH]c1=O ; "+C8F17OH" ; O=c1ccn(C2OC(CO)C(O)C2OCCC(CO)NC(F)(F)C(F)(F)C(F)(F)C(F)(F)C(F)(F)C(F)(F)C(F)(F)C(F)(F)F)c(=O)[nH]1 ; O=c1ccn(C2OC(CO)C(O)C2OCC[C@@H](CO)NC(F)(F)C(F)(F)C(F)(F)C(F)(F)C(F)(F)C(F)(F)C(F)(F)C(F)(F)F)c(=O)[nH]1]

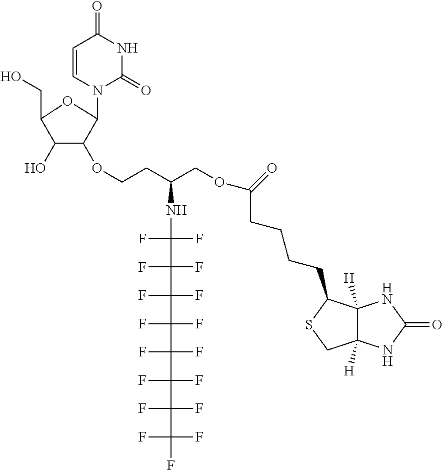 [SMILES: O=C1N[C@H]2[C@H](CS[C@H]2CCCCC(=O)OC[C@H](CCOC2C(O)C(CO)OC2n2ccc(=O)[nH]c2=O)NC(F)(F)C(F)(F)C(F)(F)C(F)(F)C(F)(F)C(F)(F)C(F)(F)C(F)(F)F)N1]

### Sample Embodiment 6: Synthesis of a Perfluorcarbylated Nucleoside with Fluorescent Dye

A fluorescein building block substituted with a reactive amino function was synthesized, thus enabling linking to the nucleoside derivate via an amide bond. 5-nitrofluoroscein was prepared from 4-nitrophthalic acid and resorcinol. The condensation reaction yielded a mixture of a 6- and 5-nitrofluorescein, and the 6-nitrofluorescein was isolated using fractional crystallization. During the subsequent synthesis, the nitro function was reduced to the amino group. The carboxyl group in the compound was converted into a methyl ester. The resulting compound was acylated with succinic anhydride, yielding the corresponding amide, as described in the scheme.

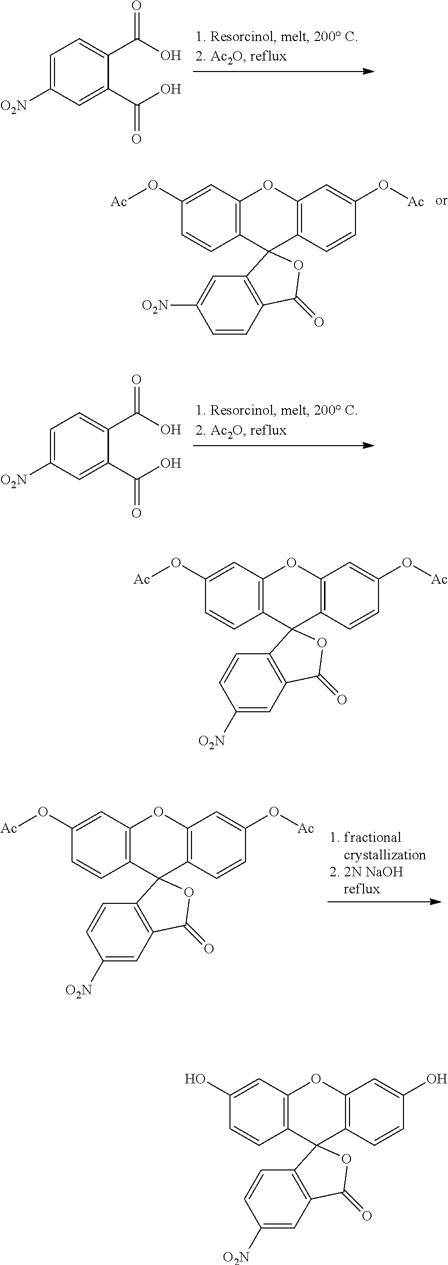

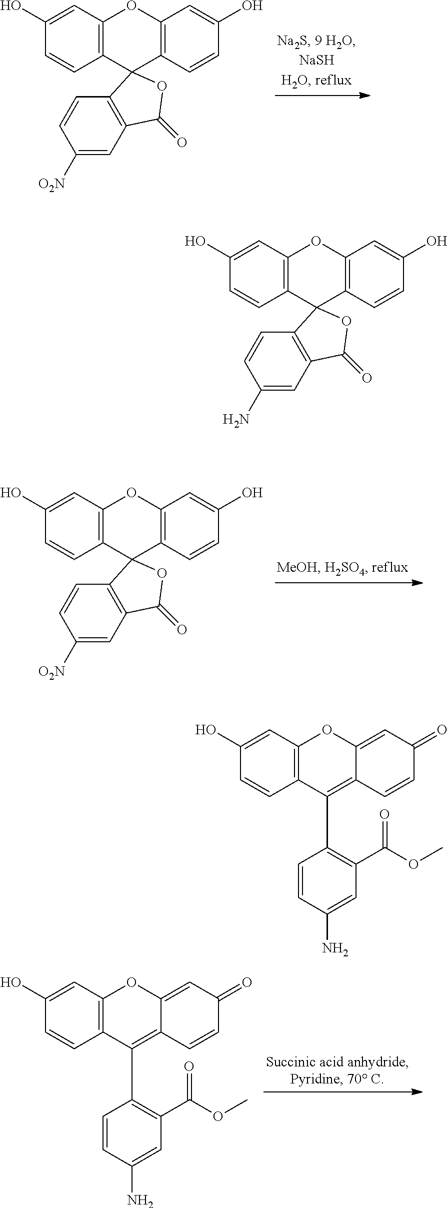 [SMILES: O=C1OC2(c3ccc(O)cc3Oc3cc(O)ccc32)c2ccc([N+](=O)[O-])cc21 ; Nc1ccc2c(c1)C(=O)OC21c2ccc(O)cc2Oc2cc(O)ccc21 ; O=C1OC2(c3ccc(O)cc3Oc3cc(O)ccc32)c2ccc([N+](=O)[O-])cc21 ; COC(=O)c1cc(N)ccc1-c1c2ccc(=O)cc-2oc2cc(O)ccc12 ; COC(=O)c1cc(N)ccc1-c1c2ccc(=O)cc-2oc2cc(O)ccc12]

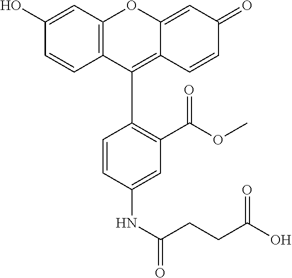 [SMILES: COC(=O)c1cc(NC(=O)CCC(=O)O)ccc1-c1c2ccc(=O)cc-2oc2cc(O)ccc12]

The free carboxyl function was esterified with a hydroxyl function of the perfluoroalkylated nucleotide.

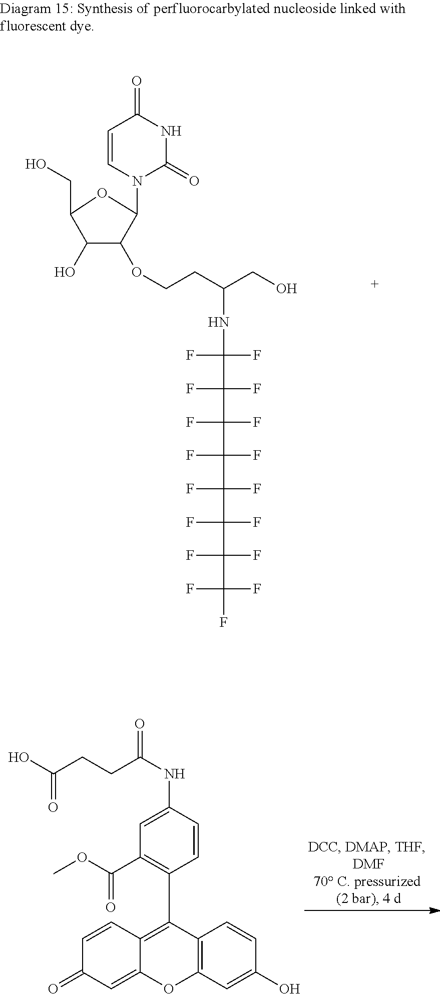 [SMILES: O=c1ccn(C2OC(CO)C(O)C2OCCC(CO)NC(F)(F)C(F)(F)C(F)(F)C(F)(F)C(F)(F)C(F)(F)C(F)(F)C(F)(F)F)c(=O)[nH]1 ; COC(=O)c1cc(NC(=O)CCC(=O)O)ccc1-c1c2ccc(=O)cc-2oc2cc(O)ccc12]

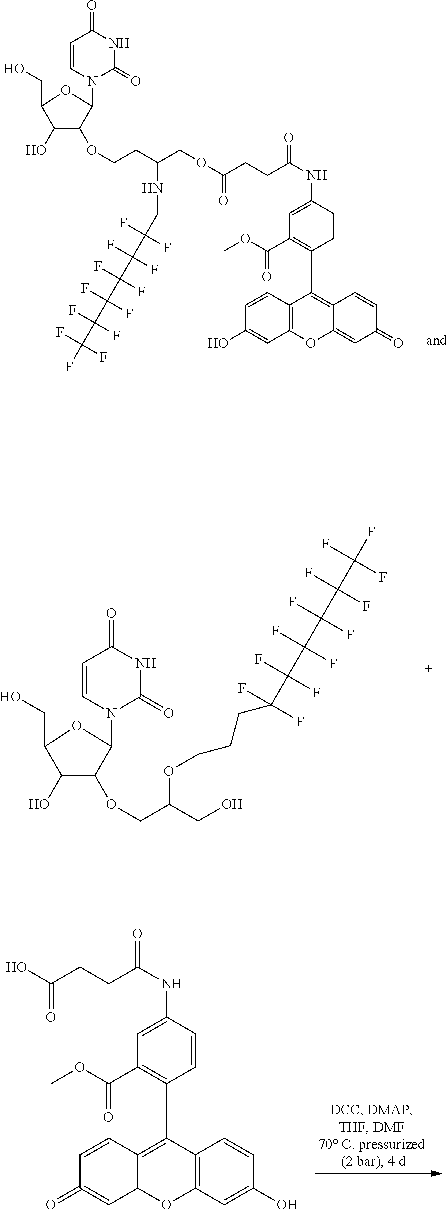

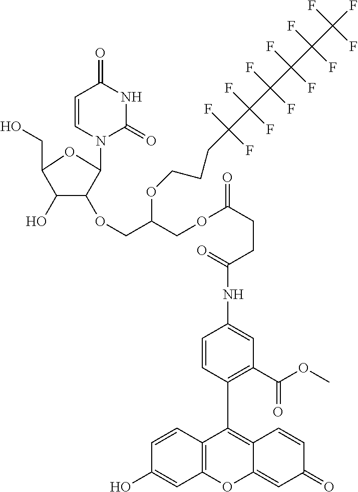 [SMILES: COC(=O)c1cc(NC(=O)CCC(=O)OCC(COC2C(O)C(CO)OC2n2ccc(=O)[nH]c2=O)OCCCC(F)(F)C(F)(F)C(F)(F)C(F)(F)C(F)(F)C(F)(F)F)ccc1-c1c2ccc(=O)cc-2oc2cc(O)ccc12]

### Sample Embodiment 7: Synthesis of a Perfluorocarbylated Nucleoside with Recognition Sequence and Fluorescent Dye

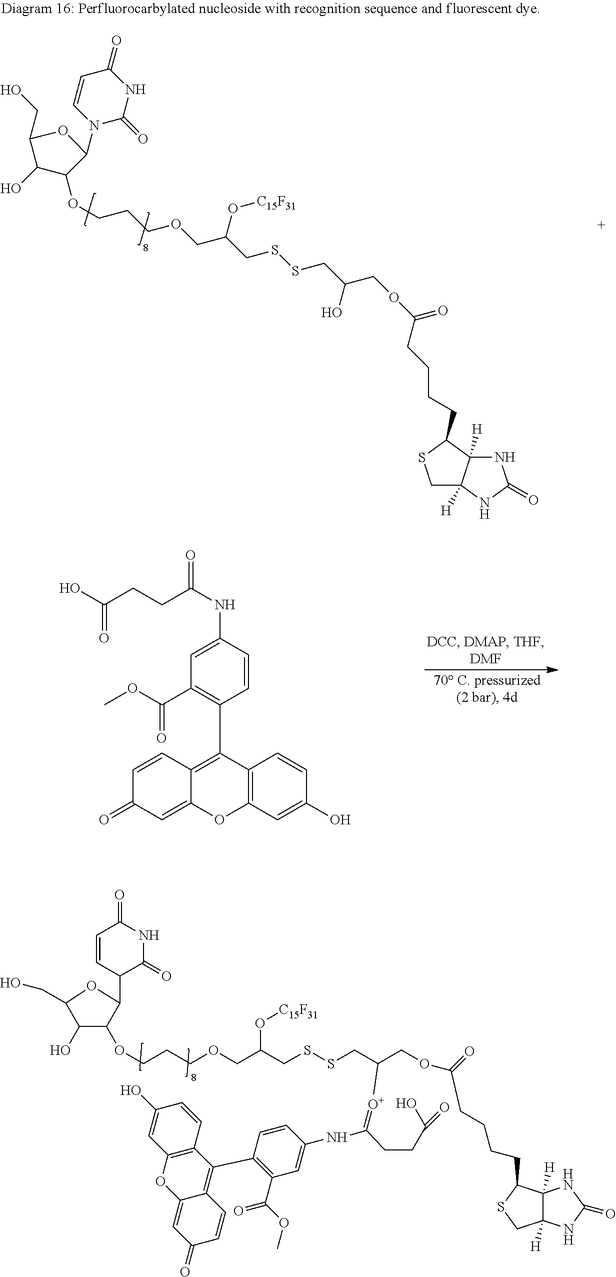

### Sample Embodiment 8: Alternative Synthesis of Perfluorocarbylated Oligonucleotides and Nucleic Acids

The perfluorocarbylation of entire oligomers and nucleic acids is also possible, for example via acid-catalyzed methods using fluoric acid; or the polymerization of perfluorocarbylated nucleotides is possible using polymerases (Polymerase Chain Reaction).

### Sample Embodiment 9: Perfluorocarbylation of Modified Nucleic Acid Building Blocks

Nucleic acids can be derivatized to stabilize or eliminate the electrical charge, for example by means of phosphorothioates, electrically neutral methyl phosphonate derivatives, electrically neutral dimethyl sulfone derivatives or derivatization at the 2′ carbon atom of the ribose. Similar to non modified nucleic acid building blocks, the possibilities for perfluorocarbyl derivatisation are on the modified sugar-phosphate backbone and at the bases (see Diagram 17). For perfluorocarbyl derivatisation paths, see Synthesis of perfluorocarbylated nucleosides, nucleotides and perfluorocarbylated oligonucleotides (prior sample embodiments).

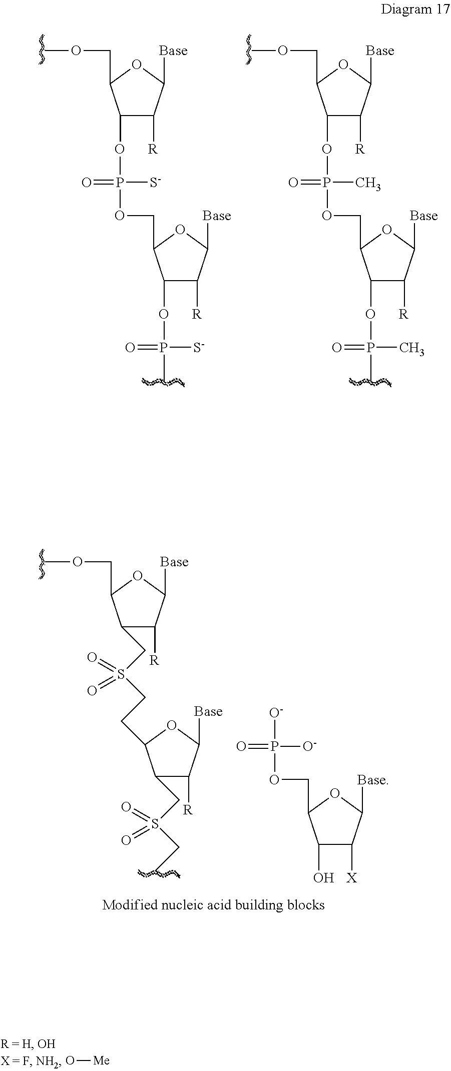

### Sample Embodiment 10: Synthesis of Perfluorcarbylated Peptide Nucleic Acid Monomers in the Form of Alanyl Nucleoamino Acids

Peptide nucleic acid monomers are nucleotide analogs in which the ribose-phosphodiester backbone has been replaced by a peptidic backbone that is based on N-(2-aminoethyl)glycine subunits or other peptide units. Here, the perfluorcarbylated bases are linked to the backbone via a carboxymethylene unit. While perfluorocarbylation at the NH₂ groups of the nucleobases exhibits no impact on the incorporation into an oligomer, the perfluorocarbylation at the amino-terminal and carboxy-terminal ends of the peptide units has the effect of halting synthesis. The synthesis of alanyl nucleoamino acids starts from Boc-L-serine and Boc-D-serine that have been converted into Boc-L-serine lactone. The Boc-serine lactone reacts by a nucleophilic ring opening in the presence of benzyloxycarbonyl-protected cytosine and the guanine precursor 2-amino-6-chloropurine to give the Boc-L-AlaG-OH or Boc-D-AlaG-OH and Boc-AlaCZ-OH or its enantiomer. The protective group benzyloxycarbonyl at the exocyclic amino function of the cytosine is necessary for the subsequent peptide solid-phase synthesis. The guanine does not require any protection, since the exocyclic amino group exhibits very poor nucleophilicity.

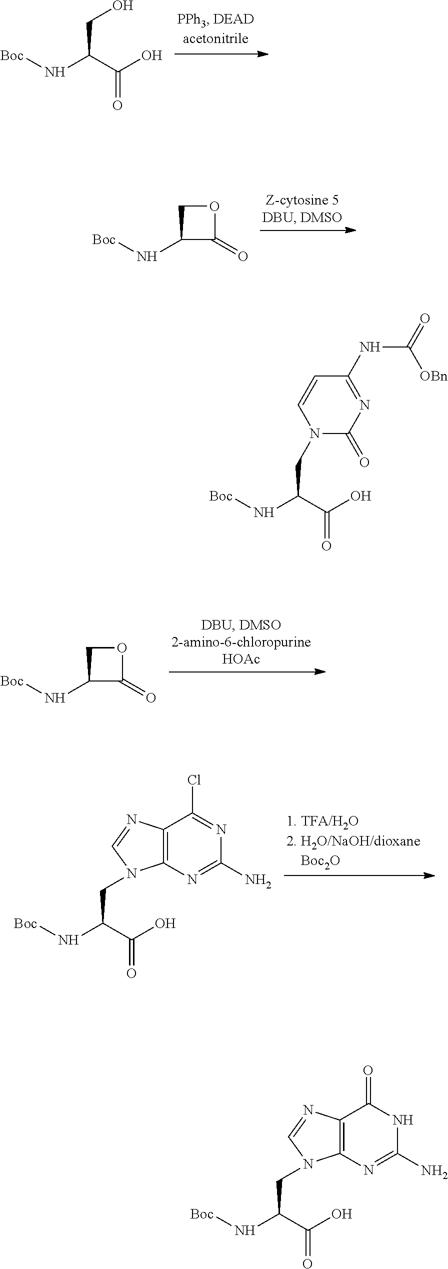 [SMILES: CC(C)(C)OC(=O)N[C@@H](CO)C(=O)O ; CC(C)(C)OC(=O)N[C@H]1COC1=O ; CC(C)(C)OC(=O)N[C@@H](Cn1ccc(NC(=O)OCc2ccccc2)nc1=O)C(=O)O ; CC(C)(C)OC(=O)N[C@H]1COC1=O ; CC(C)(C)OC(=O)N[C@@H](Cn1cnc2c(Cl)nc(N)nc21)C(=O)O ; CC(C)(C)OC(=O)N[C@@H](Cn1cnc2c(=O)[nH]c(N)nc21)C(=O)O]

Boc-L-aspartic acid benzyl ester or Boc-D-aspartic acid benzyl ester was used as the starting material for the synthesis of homoalanyl-nucleoamino acids. The side chains were reduced with BH3-THF to give alcohols which were brominated via an Appel reaction into N-Boc-D-γ-bromine-homoalanyl benzyl ester or N-Boc-D-Y-bromine-homoalanyl benzyl ester.

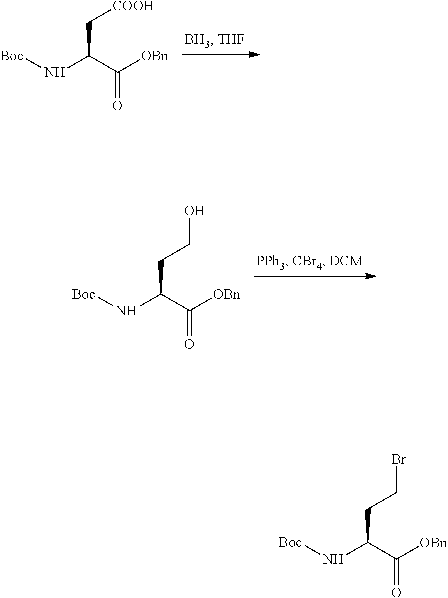 [SMILES: CC(C)(C)OC(=O)N[C@@H](CC(=O)O)C(=O)OCc1ccccc1 ; CC(C)(C)OC(=O)N[C@@H](CCO)C(=O)OCc1ccccc1 ; CC(C)(C)OC(=O)N[C@@H](CCBr)C(=O)OCc1ccccc1]

In the presence of K₂CO₃, nucleophilic substitutions of the bromide with benzyloxycarbonyl-protected cytosine and 2-amino-6-chloropurine were performed. In the next step, TFA/H₂O hydrolysis was carried out with concomitant removal of the Boc protective group. Following hydrogenation with PdO/H₂ then removed the benzyl groups. This was followed by protection of the amino group with Boc anhydride into Boc-L-HalG-OH or Boc-D-HalG-OH.

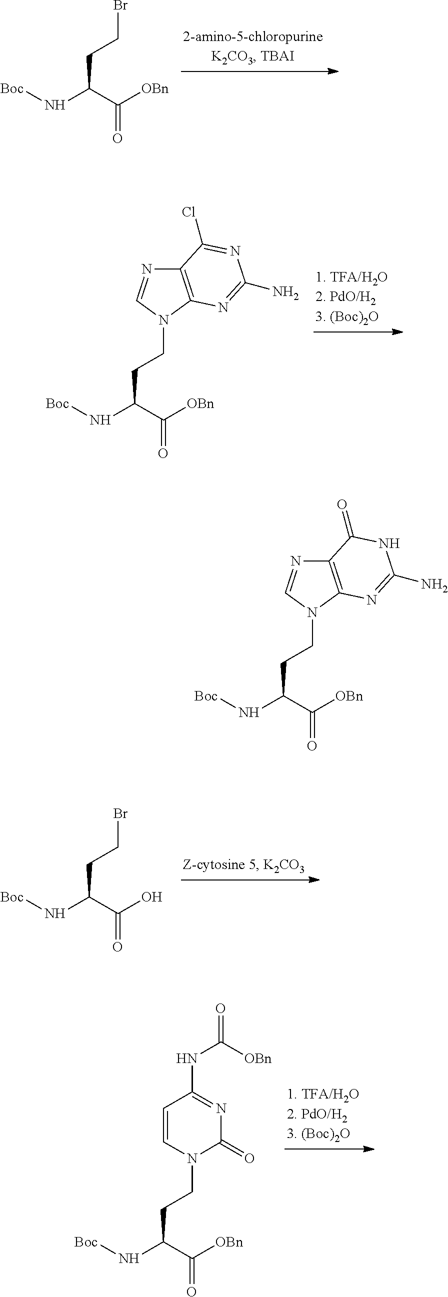 [SMILES: CC(C)(C)OC(=O)N[C@@H](CCBr)C(=O)OCc1ccccc1 ; CC(C)(C)OC(=O)N[C@@H](CCn1cnc2c(Cl)nc(N)nc21)C(=O)OCc1ccccc1 ; CC(C)(C)OC(=O)N[C@@H](CCn1cnc2c(=O)[nH]c(N)nc21)C(=O)OCc1ccccc1 ; CC(C)(C)OC(=O)N[C@@H](CCBr)C(=O)O ; CC(C)(C)OC(=O)N[C@@H](CCn1ccc(NC(=O)OCc2ccccc2)nc1=O)C(=O)OCc1ccccc1]

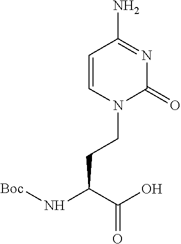 [SMILES: CC(C)(C)OC(=O)N[C@@H](CCn1ccc(N)nc1=O)C(=O)O]

The perfluorocarbylation of the monomers is done by means of Williamson's ether synthesis using K₂CO₃/acetone and a perfluorocarbyl halide over 48 hours. All of the accessible NH₂ and OH groups in the nucleobases and the peptide building blocks were perfluorocarbylated. One example of this perfluorocarbylation is shown here using the example of C₈F₁₇I.

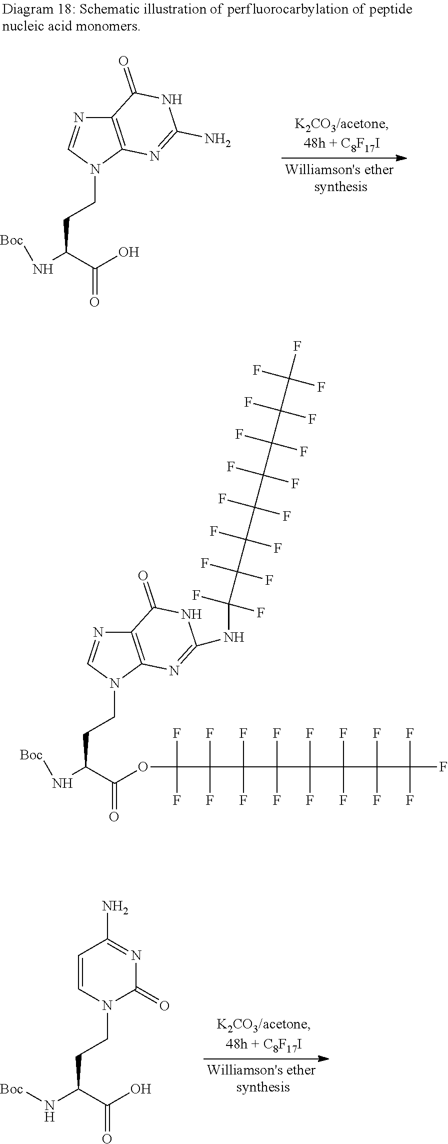 [SMILES: CC(C)(C)OC(=O)N[C@@H](CCn1cnc2c(=O)[nH]c(N)nc21)C(=O)O ; CC(C)(C)OC(=O)N[C@@H](CCn1cnc2c(=O)[nH]c(NC(F)(F)C(F)(F)C(F)(F)C(F)(F)C(F)(F)C(F)(F)C(F)(F)C(F)(F)F)nc21)C(=O)OC(F)(F)C(F)(F)C(F)(F)C(F)(F)C(F)(F)C(F)(F)C(F)(F)C(F)(F)F ; CC(C)(C)OC(=O)N[C@@H](CCn1ccc(N)nc1=O)C(=O)O]

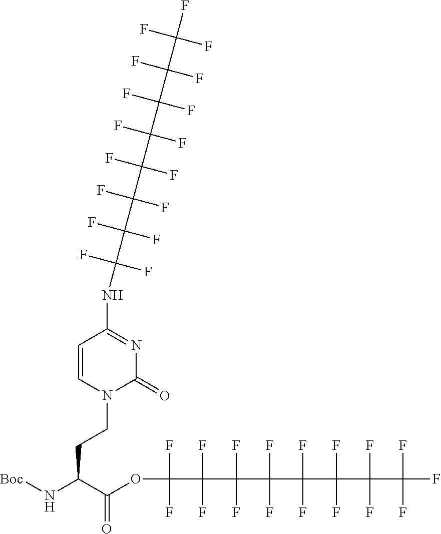 [SMILES: CC(C)(C)OC(=O)N[C@@H](CCn1ccc(NC(F)(F)C(F)(F)C(F)(F)C(F)(F)C(F)(F)C(F)(F)C(F)(F)C(F)(F)F)nc1=O)C(=O)OC(F)(F)C(F)(F)C(F)(F)C(F)(F)C(F)(F)C(F)(F)C(F)(F)C(F)(F)F]

Another possibility is the masking of the OH groups of the peptide fraction before perfluorocarbylation takes place. Under those circumstances, only the NH₂ groups of the nucleobases are perfluorocarbylated. To achieve this, the hydroxy groups must be protected with ditertbutylsilylditriflate. After perfluorocarbylation, these OH groups are liberated in order to be available for peptide synthesis. Using these monomers, an oligomer synthesis is possible in which perfluorocarbylated monomers are already incorporated. However, due to the additional reaction steps that are required, it appears to be easier to first synthesize and then perfluorocarbylate the oligomer.

Additional perfluorocarbylations are listed, as examples:

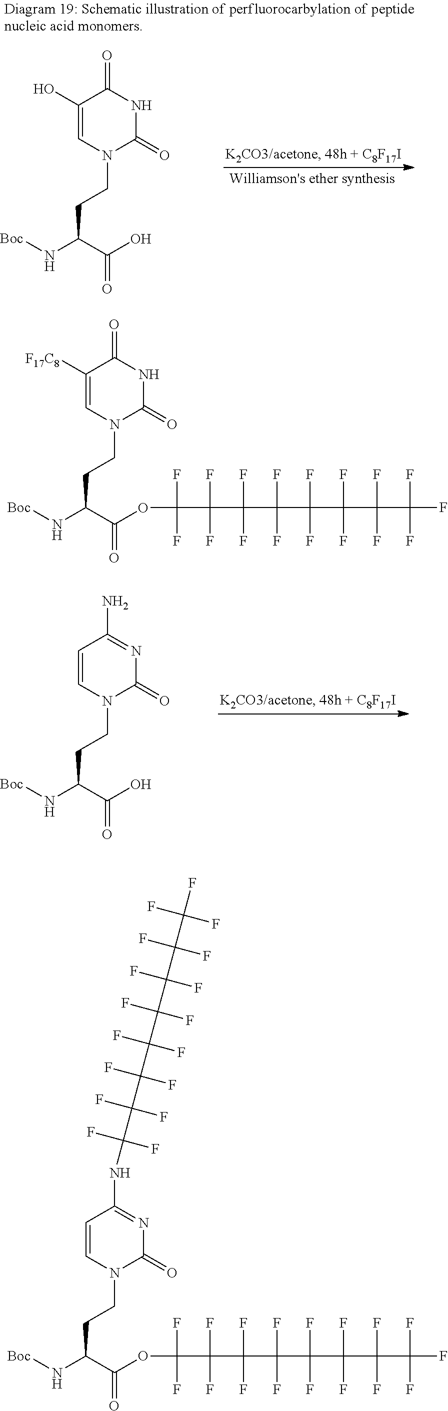

### Sample Embodiment 11: Synthesis of Perfluorocarbylated Peptide Nucleic Acid Oligomers and Peptide Nucleic Acids

In the case of peptide nucleic acids, the entire ribose-phosphodiester backbone was replaced by a peptidic backbone based on N-(2-aminoethyl)glycine subunits or other peptide units. The perfluorocarbylated bases were linked here to the backbone via a carboxymethylene unit. Next, the nucleobases were linked to the peptide units. Then the monomers were linked to oligomers in solid-phase synthesis. It has proven simpler to perform the perfluorocarbylation steps only after the synthesis of the oligomers. The perfluorocarbylation steps are the same as those reaction steps used in the perfluorocarbylation of the monomers.

The synthesis of alanyl-nucleoamino acids starts from N-Boc-L-serine and Boc-D-serine that have been converted into N-Boc-L-serine lactone or N-Boc-D-serine lactone. The Boc-serine lactone reacts by a nucleophilic ring opening in the presence of the benzyloxycarbonyl-protected cytosine and the guanine precursor 2-amino-6-chlorepurine into Boc-L-AlaG-OH or Boc-D-AlaG-OH and Boc-AlaCZ-OH or its enantiomer. The protective group benzyloxycarbonyl on the exocyclic amino function of the cytosine is necessary for the subsequent peptide solid-phase synthesis. The guanine does not require any protection, since the exocyclic amino group exhibits very poor nucleophilicity.

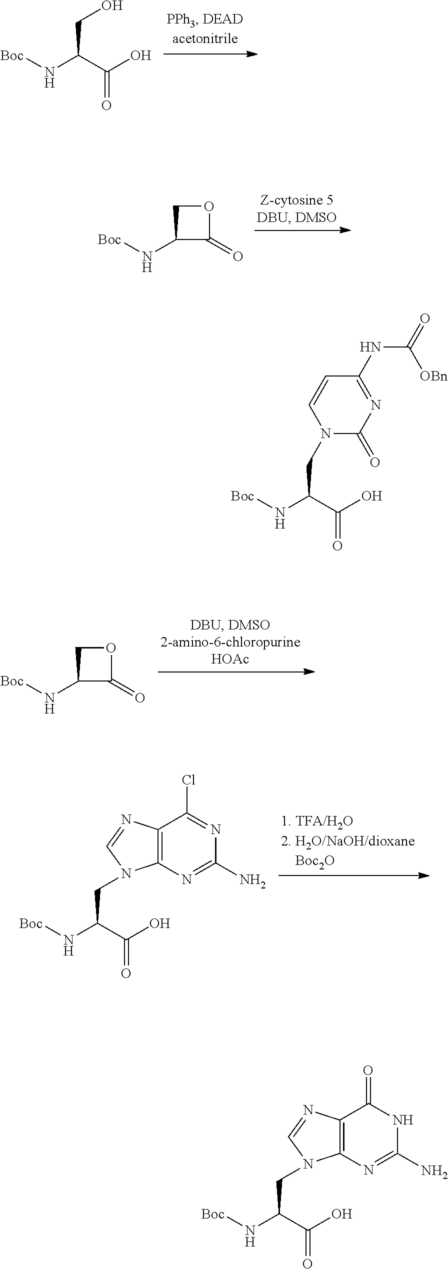 [SMILES: CC(C)(C)OC(=O)N[C@@H](CO)C(=O)O ; CC(C)(C)OC(=O)N[C@H]1COC1=O ; CC(C)(C)OC(=O)N[C@@H](Cn1ccc(NC(=O)OCc2ccccc2)nc1=O)C(=O)O ; CC(C)(C)OC(=O)N[C@H]1COC1=O ; CC(C)(C)OC(=O)N[C@@H](Cn1cnc2c(Cl)nc(N)nc21)C(=O)O ; CC(C)(C)OC(=O)N[C@@H](Cn1cnc2c(=O)[nH]c(N)nc21)C(=O)O]

Boc-L-aspartic acid benzyl ester or Boc-D-aspartic acid benzyl ester was used as the starting material for the synthesis of homoalanyl-nucleoamino acids. The side chains were reduced with BH3-THF to give alcohols which were brominated via an Appel reaction into N-Boc-L-γ-bromine-homoalanyl benzyl ester or N-Boc-D-γ-bromine-homoalanyl benzyl ester.

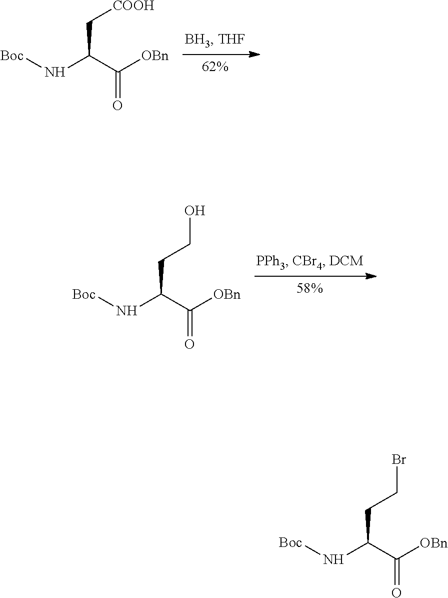 [SMILES: CC(C)(C)OC(=O)N[C@@H](CC(=O)O)C(=O)OCc1ccccc1 ; CC(C)(C)OC(=O)N[C@@H](CCO)C(=O)OCc1ccccc1 ; CC(C)(C)OC(=O)N[C@@H](CCBr)C(=O)OCc1ccccc1]

In the presence of K₂CO₃, nucleophilic substitutions of the bromide with benzyloxycarbonyl-protected cytosine and 2-amino-6-chloropurine were performed. In the next step, TFA/H₂O was hydrolyzed, with the Boc protective group being removed simultaneously. Following hydrogenation with PdO-H₂ then removed the benzyl groups. This was followed by protection of the amino group with Boc anhydride into Boc-L-HalG-OH or Boc-D-HalG-OH.

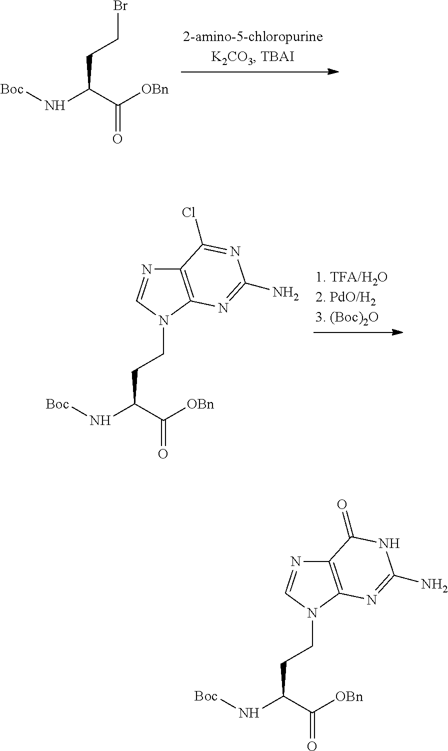 [SMILES: CC(C)(C)OC(=O)N[C@@H](CCBr)C(=O)OCc1ccccc1 ; CC(C)(C)OC(=O)N[C@@H](CCn1cnc2c(Cl)nc(N)nc21)C(=O)OCc1ccccc1 ; CC(C)(C)OC(=O)N[C@@H](CCn1cnc2c(=O)[nH]c(N)nc21)C(=O)OCc1ccccc1]

As the obtained benzyloxycarbonyl protective group at the cytosine base was the desired result, no hydrogenolytic cleavage of the benzyl group can occur at this position. A basic saponification was therefore performed with NaOH/dioxane/H₂O.

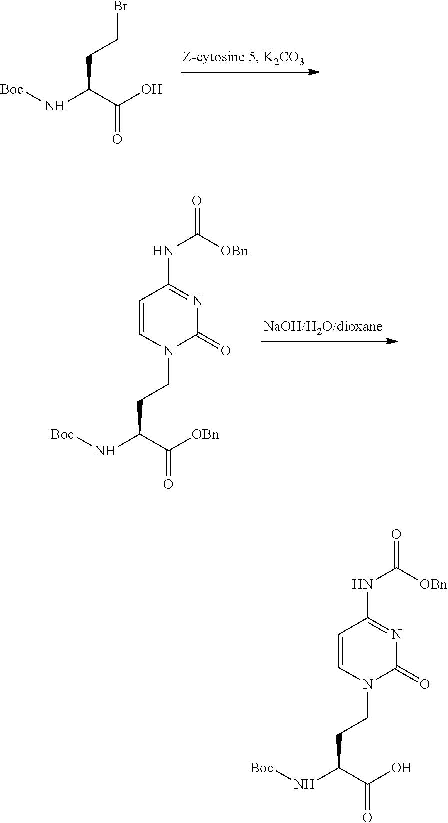 [SMILES: CC(C)(C)OC(=O)N[C@@H](CCBr)C(=O)O ; CC(C)(C)OC(=O)N[C@@H](CCn1ccc(NC(=O)OCc2ccccc2)nc1=O)C(=O)OCc1ccccc1 ; CC(C)(C)OC(=O)N[C@@H](CCn1ccc(NC(=O)OCc2ccccc2)nc1=O)C(=O)O]

The synthesis of peptide nucleic acids is performed analogously to the synthesis of peptides. The synthesis is performed on solid-state systems. The synthesis can either be carried out using Boc synthesis methods or the Fmoc synthesis method. In this case, the Boc synthesis method was chosen:

HBTU (N-(1-H-benzotriazole-1-yl)-1,1,3,3-tetramethyluronium-hexafluorophosphate) or HOBt (1-hydroxybenzotriazole) were used as coupling reagents for the amino acids. The homoalanyl nucleoamino acids were activated with HATU or HOAt, respectively. MBHA-PS resin, overlaid with Boc-L-Lys(2-CI—Z)—OH, was used as the solid state. Depending on the amino acid, coupling took place between 35 minutes and two hours. The protective groups of the side chains were selected such that they could be removed simultaneously with the acidic cleaving-off from resin. For lysine, the side chain was protected with a (2-CI—Z), for glutaminic acid with a (OBn) and for tyrosine with a (2-Br—Z) group.

1. Deprotection: 5% m-cresol in TFA (1×5 min, 1×10 min); 2. Washing: DCM/NMP (5×)+pyridine;
1. Coupling: 5.0 eq. Boc-Hal-OH or 5.0 eq. Boc-AS-OH, 4.5 eq. HATU or HBTU, 5.0 eq. HOAt or HOBt 12 eq. DIPEA, NMP; 2. Washing: DCM/NMP (5×), 10% piperidine in NMP (3×), DCM/NMP (5×);

1. Capping: Ac20/DIPEA/NMP (1:1:8), (2×5 min); 2. Washing: DCM/NMP (5×), 10% piperidine in NMP (3×), DCM/NMP (5×); Cleavage: TFA/TFMSA/m-cresol (8:1:1).

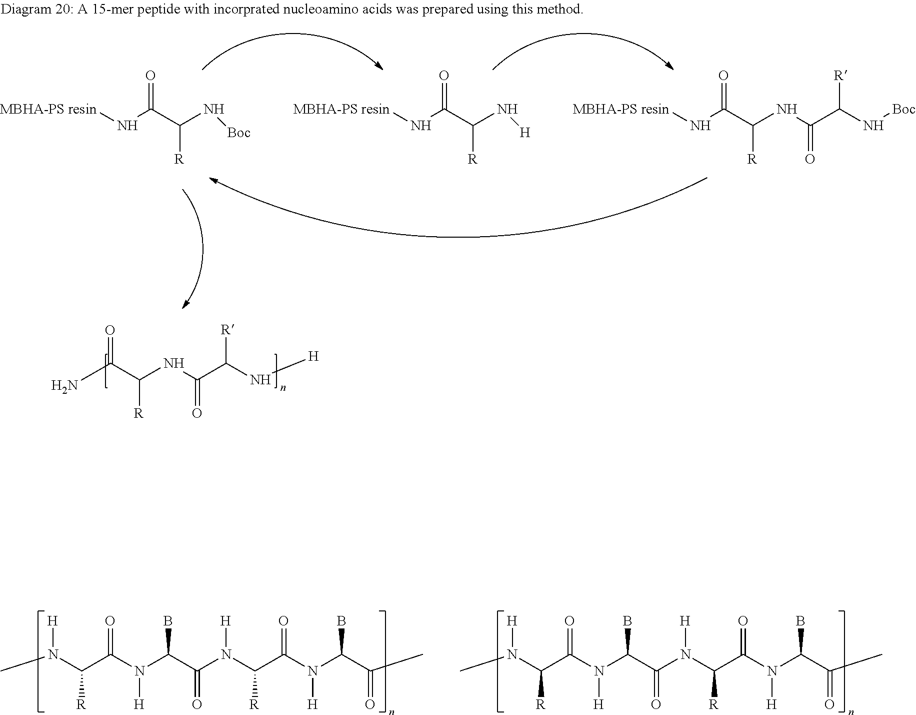

This was followed by Williamson's ether synthesis using of K₂CO₃/acetone and a functionalized perfluorocarbon molecule for 48 hours. The perfluorocarbylation occurred on all accessible NH₂ and OH groups, with the reaction occurring analogously to the perfluorocarbylation of individual nucleobases and peptide nucleic acid monomers (as described above). By varying the reaction time (2 h to 72 h), the degree of perfluorocarbylation was able to be reduced or increased. (R, R′) nucleobases, (B) Thyminyl.

### Sample Embodiment 12: Selection of the Predetermined Breaking Points

The following predetermined breaking points were used here: The perfluorocarbylated mRNA complexes are absorbed by endocytosis and packed in lysosomes. During this process, a substantial jump in pH from 7.4 to 7.2 occurs in the extracellular space to up to 4.0 in the lysosome that is caused by an ATP-dependent proton pump (Serresi et al. 2009). This low pH value of 4.0 is crucial for the selection of the predetermined breaking point. There is a series of acid-labile predetermined breaking points that are valuable of consideration for the PFC system (Warnecke, 2008, Warnecke 2010). However, glycosidic bonds at the 2′ position hydrolyze at low pH values; see diagram 21.

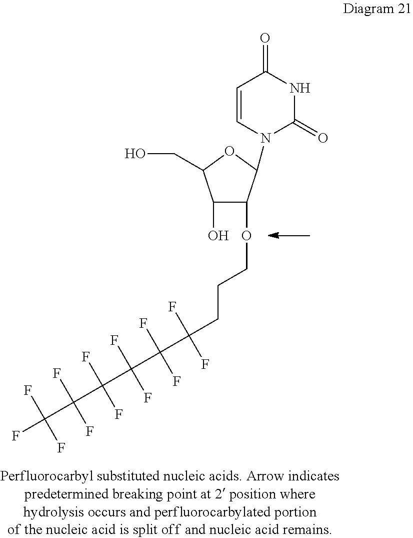 [SMILES: O=c1ccn(C2OC(CO)C(O)C2OCCCC(F)(F)C(F)(F)C(F)(F)C(F)(F)C(F)(F)C(F)(F)F)c(=O)[nH]1]

Perfluorohydrocarbyl groups protect the molecule from hydrolysis by hydrolases. Only chemical hydrolysis can occur. The cleaved perfluorohydrocarbyl group leads to inert molecules which do not react with cell molecules. However, predetermined breaking points that break in the cytoplasm are useful, too (approx. pH values=7).

### Sample Embodiment 13: Alternative Synthesis of Perfluorocarbylated Oligonucleotides and Nucleic Acids

The perfluorocarbylation of entire oligomers and nucleic acids is also possible, for example using acid-catalytic methods with fluoric acid or by polymerization of perfluorocarbylated nucleotides using polymerases (Polymerase Chain Reaction).

### Sample Embodiment 14: Preparation of an Emulsion

A) To achieve certain particle sizes, it may be necessary to use surfactants. Pluronic F-68 was used here: 5 mg Pluronic F-68 is dissolved in 10 ml distilled water. 0.5 ml of a functionalized perfluorocarbylated/mRNA solution (1.0 g/1.0 microliter) was added to this. Sonification is then performed for 3 cycles, intensity 60. The obtained emulsion is then centrifuged (1200 RPM/5 min) in order to deposit the excessively large particles. Particles with a particle size of 50-100 nanometers are found in the supernatant above. These are used.

B) Solvent emulsion: 0.5 ml of a functionalized perfluorocarbylated/mRNA solution (1.0 g/1.0 microliter) is added to 2 ml tetrahydrofuran to form a solution. This is then brought to 10 ml using distilled water. The emulsion obtained is then centrifuged (1200 RPM/5 min) in order to deposit the excessively large particles. Particles with a particle size of 50-100 nanometers are found in the supernatant above. These are used.

C) 0.5 ml of a functionalized perfluorocarbylated/mRNA solution (1.0 g/1.0 microliter) is added to 10 ml distilled water. Sonification is then performed for 3 cycles, intensity 60. The obtained emulsion is then centrifuged (1200 RPM/5 min) in order to deposit the excessively large particles. Particles with a particle size of 50-100 nanometers are found in the supernatant above. These are used.

### Sample Embodiment 15: Preparation of an Artificial mRNA with the Therapeutic Sequence for Treating an Acquired Genetic Disease

The artificial perfluorocarbylated mRNA is prepared as described above. The predetermined breaking point of this system is a glycosidic bond. In addition, the GC content of the artificial mRNA is increased while maintaining the same coding information, which increases the life span (resistance to RNAses).

Compounds of artificial mRNA and functionalized perfluorocarbon have both hydrophilic and hydrophobic characteristics and require no additional surfactant. the preparation of the emulsion is done as described under C) of sample embodiment 15. The emulsion prepared in a buffer is processed by an apparatus into an aerosol that is applied as an inhalation spray and gets into the bloodstream of the body via the lung. The compound circulates in the blood and is absorbed non-specifically through endocytosis/pinocytosis. The breakage of the predetermined breaking points occurs in the endosomes and lysosomes of the cell through chemical hydrolysis. The released mRNA and the released perfluorocarbon molecules are released by the endosome or by the lysosome into the cytoplasm. The translation of the mRNA and the formation of the therapeutic proteins occur in the cytoplasm. The transport system (perfluorocarbylated fragment) is inert and cannot react with cell molecules. Due to its vapor pressure and other physical properties, the transport system is excreted via the lung and kidney function.

### Sample Embodiment 16: Release of Therapeutic siRNA in Cancer Treatment

The preparation of the siRNA and the linking of transferrin to the system is done as described above, as is the preparation of the emulsion. This emulsion is administered intravenously. By virtue of the ligand transferrin, the particles are absorbed especially and strongly in tumor cells, which enables treatment with siRNA in specific target cells. The predetermined breaking points are broken in the endosomes or the lysosomes by chemical hydrolysis. siRNA and perfluorocarbon molecules are released into the cytoplasm. The perfluorocarbon molecules are excreted via the lung and kidney function.

### Sample Embodiment 17: A Therapeutic Vaccination Against HPV Type 16

siRNA for shutting down a specific gene expression of HVP type 16 and emulsion thereof is prepared as described above. This emulsion is processed in a buffer into a tincture that is applied to the mucosa. The complexes penetrate into the tissue and are absorbed by the cells through endocytosis/pinocytosis. The path of action of the siRNA and path of elimination of the perfluorocarbylated fragments are as described above.

### Sample Embodiment 18: Detection of the Absorption of Perfluorocarbylated Nucleic Acid into the Cell

To follow the path of the perfluorocarbylated nucleic acids into the cell, the following compound bearing a rhodamine label was used:

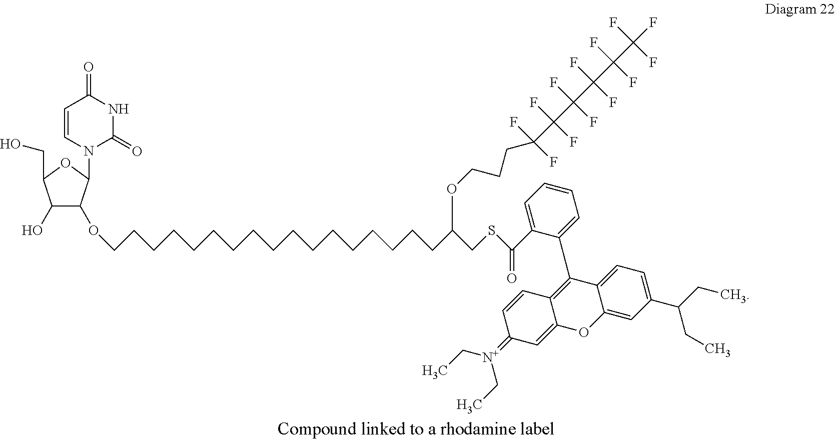 [SMILES: CCC(CC)c1ccc2c(-c3ccccc3C(=O)SCC(CCCCCCCCCCCCCCCCCOC3C(O)C(CO)OC3n3ccc(=O)[nH]c3=O)OCCCC(F)(F)C(F)(F)C(F)(F)C(F)(F)C(F)(F)C(F)(F)F)c3ccc(=[N+](CC)CC)cc-3oc2c1]

The compound was first dissolved in tetrahydrofuran (THF), and this solution was subsequently titrated in isopropanol. The obtained particles had an average size of 50 nm.

Cells of line HEK 293 were used as cell culture. Transfection was performed one day after cell seeding. As a control, a transfection was performed with pure rhodamine particles of equivalent rhodamine concentration to test the influence of rhodamine on the internalization of perfluorocarbylated nucleic acids. Immediately after transfection, the cell culture medium of the cells incubated with rhodamine-labeled perfluorocarbylated nucleic acids appeared clear and unchanged. The cell culture medium of cells incubated with rhodamine only appeared slightly cloudy and reddish. One explanation for this is that the rhodamine is bound to the particles of perfluorocarbylated nucleic acids whereby particles sinks to the bottom of the cell culture dishes, whereas the dye is completely dissolved in the medium with pure rhodamine.

20 minutes after transfection, a homogeneous coloration could be observed over the cell surface in the cells incubated with rhodamine labeled perfluorocarbylated nucleic acids. In the cells incubated with pure rhodamine, no defined coloration could be seen at this time point.

24 hours after transfection, the coloration of the cells with rhodamine-labeled perfluorocarbylated nucleic acids had changed compared to the 20 minute mark: The uniformly homogeneous coloration on the cell surface could no longer be observed. Instead, a granular coloration was observed whose vesicles had elevated color intensity, whereas the intermediate spaces hardly exhibited any coloration.

During the investigation of this process using images on the confocal microscope (see FIG. 1), it became evident that the perfluorocarbylated nucleic acids were transported in the endosomes and lysosomes. The endosomes and lysosomes filled with perfluorocarbylated nucleic acids were detected throughout the cell cytoplasm. The particles could not be found in the nucleus (nucleus localization sequence or cell division required). In addition, it was observed that a quantity of particles released from the endosomes/lysosomes was already located in the cytoplasm, which was apparent in the diffuse coloration outside of the vesicle.

In contrast, when cells were incubated with pure rhodamine, a coloration was also observed after 24 hours. The coloration was substantially weaker and diffuse on the cell surface compared to the cytoplasm where the coloration was stronger and granular.

The transfected cells were also studied using FACS (Fluorescence Activated Cell Sorting) (see FIG. 2). The FACS studies were conducted on cells transfected with pure rhodamine (control) and with perfluorocarbylated nucleic acids. The results of confocal microscopy were confirmed. perfluorocarbylated nucleic acids are indeed absorbed by the cell, packed in endosomes and lysosomes, and released by these into the cytoplasm.

As a result of the high effectiveness of the absorption of perfluorocarbylated nucleic acids into the cell, this method constitutes a true alternative to virally mediated gene transfer and to other methods for transferring nucleic acids and analogs thereof (modified nucleic acids, peptide nucleic acids) into the cell.

What is claimed is:

1. An oligonucleotide or a nucleic acid covalently bonded to a perfluorocarbyl group according to the general formula ABE, wherein:
A is a perfluorocarbyl group selected from the group consisting of:
perfluorinated straight or branched aliphatic alkanes,
perfluorinated straight or branched alkenes,
perfluorinated straight or branched alkynes, and
cyclic, optionally aromatic, perfluorocarbons, in which all of the H-atoms are substituted by F-atoms,
B is a covalent bond, and
E is an oligonucleotide or a nucleic acid,
wherein B is selected from the group consisting of:
(i) a covalent bond between the perfluorocarbyl group A and a C atom at the 2′ position of one or more sugars of the oligonucleotide or the nucleic acid, wherein B is an ester bond that forms a structure A-CO—O-E, an amide bond that forms a structure A-CO—NH-E, or an ether bond that forms a structure A-O-E, wherein E is the C atom at the 2′ position of said one or more sugars;
(ii) a covalent bond between the perfluorocarbyl group A and a C atom of a heterocyclic ring of one or more nucleobases of the oligonucleotide or the nucleic acid, wherein B is an amide bond that forms a structure A-CO—NH-E, an ether bond that forms a structure A-O-E, or an amine bond that forms a structure A-NH-E, wherein E is the C atom of said heterocyclic ring of one or more nucleobases; and
(iii) a covalent bond between the perfluorocarbyl group A and an O atom of one or more phosphate groups of the oligonucleotide or the nucleic acid, wherein B is a phosphate ester bond that forms a structure A-CH₂-E, or A-CO-E, wherein E is an O atom of said one or more phosphate groups.

2. The oligonucleotide or nucleic acid covalently bonded to a perfluorocarbyl group as set forth in claim 1, wherein the perfluorocarbyl group contains C₁-C₂₀₀.

3. The oligonucleotide or nucleic acid covalently bonded to a perfluorocarbyl group as set forth in claim 1, wherein the covalent bonding between the perfluorocarbyl group and the oligonucleotide or the nucleic acid is embodied in the form of an ether bond that forms a structure A-O-E, wherein E is the C atom at the 2′ position of said one or more sugars, or an ester bond that forms a structure A-CO—O-E.

4. The oligonucleotide or nucleic acid covalently bonded to a perfluorocarbyl group as set forth in claim 1, wherein the oligonucleotide or the nucleic acid comprises one or more nucleotides comprising a nucleoside structure selected from the group consisting of adenosine, guanosine, cytidine, 5-methyluridine, uridine, deoxyadenosine, deoxyguanosine, thymidine, deoxyuridine, deoxycytidine, 2-thiocytidine, N⁴-acetylcytidine, 2′-O-methylcytidine, 3-methylcytidine, 5-methylcytidine, 2-thiouridine, 4-thiouridine, pseudouridine, dihydrouridine, 5-(carboxyhydroxymethyl)-uridine, 5-carboxymethylaminomethyl-uridine, 5-methylaminomethyluridine, 5-methoxy-carbonylmethyl-uridine, 5-methoxyuridine, 2′-O-methyluridine, ribothymidine, 1-methyladenosine, 2-methyladenosine, N⁶-methyladenosine, N⁶-isopentenyladenosine, 2′-O-methyladenosine, inosine, 1-methylinosine, 1-methylguanosine, N²-methylguanosine, N²,N²-dimethylguanosine, 7-methylguanosine, 2′-O-methylguanosine, queuosine, β-D-galactosylqueuosine, β-D-mannosyl-queuosine, 2′-O-ribosyladenosinphosphate, N⁶-threonylcarbamoyladenosine, riboflavin and pantothenic acid, puromycin, acyclovir and ganciclovir.

5. The oligonucleotide or nucleic acid covalently bonded to a perfluorocarbyl group as set forth in claim 1, wherein the oligonucleotide or the nucleic acid is selected from the group consisting of single-stranded and double-stranded oligonucleotides and nucleic acids.

6. An in vitro method of non-viral transfer of at least one oligonucleotide or nucleic acid into at least one cell comprising administering a oligonucleotide or nucleic acid covalently bonded to a perfluorocarbyl group set forth in claim 1 to said cell in vitro.

7. A pharmaceutical composition comprising a oligonucleotide or nucleic acid covalently bonded to a perfluorocarbyl group as set forth in claim 1 and a pharmaceutically acceptable carrier.

8. A pharmaceutical composition as set forth in claim 7, wherein the composition is present in the form of a dispersion, suspension, emulsion or solution with an average particle size between 2 nm and 200 μm.

9. A method of non-viral transfer of at least one oligonucleotide or nucleic acid into at least one cell of a eukaryotic organism in need thereof comprising administering a oligonucleotide or nucleic acid covalently bonded to a perfluorocarbyl group set forth in claim 1 to said eukaryotic organism in need thereof.
